(19) United States
(12) Patent Application Publication
(10) Pub. No.: US 2008/0069801 A1
(43) Pub. Date: Mar. 20, 2008

(54) Methods and apparatus for using polymer-based beads and hydrogels for cardiac applications

(76) Inventors: Randall J. Lee, Hillsborough, CA (US); Francis Rauh, Plainsboro, NJ (US); Mark Maciejewski, Edina, MN (US)

Correspondence Address: CYR & ASSOCIATES, P.A., 605 U.S. Highway 169, Suite 300, Plymouth, MN, 55441, US

(21) Appl. No.: 11/818,220

(22) Filed: Jun. 13, 2007

Related U.S. Application Data
(60) Provisional application No. 60/813,184, filed on Jun. 13, 2006.

(51) Int. Cl. A61K 31/715; A61K 31/7088; A61K 31/722; A61K 38/02; A61K 38/16; A61K 45/00; A61P 9/00

(52) U.S. Cl. 424/93.1; 514/12; 514/2; 514/44; 514/54; 514/55

(57) ABSTRACT

Biopolymer beads and hydrogels are useful in the remodeling, repair and reconstruction of the heart, as well as in modification of electrical conduction in the heart. Various types of beads are useful, including beads comprising a core of alginate polymers which may or may not be bonded to peptides; beads comprising a core in which peptides are dispersed with alginate polymers, and a chitosan film ionically bonded to available alginate polymers at the surface of the core; beads comprising a core in which peptides and chitosan derivates are dispersed with alginate polymers and form alginate-peptide complexes to which the chitosan derivatives are bonded; and beads comprising a core of chitosan polymers which may or may not be bonded to peptides. The heart may also be treated with a hydrogel agent comprising alginate polymers and peptides covalently bonded to the alginate polymers.

### CROSS-REFERENCE TO RELATED APPLICATION

This application claims the benefit of U.S. Provisional Patent Application No. 60/813,184 filed Jun. 13, 2006, which hereby is incorporated herein in its entirety by reference thereto.

### BACKGROUND OF THE INVENTION

1. Field of the Invention

The present invention relates to treatment of cardiac conditions, and more particularly to methods and apparatus for using polymer beads for cardiac repair and reconstruction, and for the modification of electrical conduction in the heart.

2. Description of Related Art

Cardiovascular disease (“CVD”) is the leading cause of death in the United States, and includes various cardiac conditions generally associated with dilated cardiomyopathy, myocardial infarctions, and congestive heart failure (“CHF”). Information on the prevalence of CVD and CHF is disclosed in various publications, including Lenfant, C., “Fixing the failing heart,” Circulation 95:771-772, 1997; American Heart Association, Heart and Stroke Statistical Update, 2001; Lenfant, C., “Cardiovascular research: an NIH perspective,” Cardiovasc. Surg. 5:4-5, 1997; Cohn, J. N., et al., “Report of the National Heart, Lung, and Blood Institute Special Emphasis Panel on heart failure research,” Circulation 95:766-770, 1997.

Heart failure following a myocardial infarction (MI) is often progressive. Scar tissue formation and aneurismal thinning of the infarct region often occur in patients who survive myocardial infarctions. It is believed that the death of cardiomyocytes results in negative left ventricular (“LV”) remodeling which leads to increased wall stress in the remaining viable myocardium. This process results in a sequence of molecular, cellular, and physiological responses which lead to LV dilation. Although the exact mechanisms of heart failure are unknown, LV remodeling is generally considered an independent contributor to its progression. Negative left ventricular remodeling is believed to contribute independently to the progression of heart failure following a myocardial infarction.

Coronary artery disease and myocardial ischemia with infarction is the etiology in the majority of patients with dilated cardiomyopathies (“DCM”). DCM is characterized by left ventricular dilation, normal or decreased wall thickness, and reduced ventricular systolic function. Left ventricle (“LV”) aneurysm is a type of ischemic cardiomyopathy in which a large transmural myocardial infarction (“MI”) thins and expands over time. Aneurysm formation begins early after myocardial infarction. Further related information is disclosed in the following references: Giles, T., “Dilated Cardiomyopathy, in Heart Failure,” P. Poole-Wilson, et al., Editors, 1997, Churchill Livingstone: New York, p. 401-422; and Eaton, L. W., et al., “Regional cardiac dilatation after acute myocardial infarction: recognition by two-dimensional echocardiography, “N Engl J Med, 1979.300 (2): p. 57-62). The myocardial infarct scar can result in dyskinetic segments of the ventricle or thinning of the infarct leading to aneurysms. Either of these consequences will significantly decrease global cardiac function. Compensatory mechanisms resulting in increased mechanical stress could lead to programmed cell death of cardiocytes in the non-infarcted myocardium, resulting in cardiac remodeling; see, e.g., Cheng W, et al., “Stretch-induced programmed myocyte cell death, “J. Clin. Invest. 96: 2247-2259, 1995. Cardiac remodeling of non-infarcted myocardium has been suggested to cause ventricular dilatation which further contributes to ventricular dysfunction and the propensity for malignant arrhythmias; see, e.g., Beltrami C, et al., “Structural basis of end-stage failure in ischemic cardiomyopathy in humans,” Circulation 89: 151-163, 1994; and Olivetti G, et al., “Side-to-side slippage of myocytes participates in ventricular wall remodeling acutely after myocardial infarction in rats.” Circ. Res. 67: 23-34, 1990.).

### SUMMARY OF THE INVENTION

We have found that biopolymer beads and hydrogels are useful in the repair and reconstruction of the heart, as well as in modification of electrical conduction in the heart. Various types of beads are useful, including beads comprising a core of alginate polymers which may or may not be bonded to peptides; beads comprising a core in which peptides are dispersed with alginate polymers, and a chitosan film ionically bonded to available alginate polymers at the surface of the core; beads comprising a core in which peptides and chitosan derivates are dispersed with alginate polymers and form alginate-peptide complexes to which the chitosan derivatives are bonded; and beads comprising a core of chitosan polymers which may or may not be bonded to peptides.

In another embodiment of the invention, a cardiac infarction is treated with a bead-containing agent comprising beads having a myocardium-adhering property for lodging within the interstitial spaces to provide structural support to an infarcted myocardial region.

In another embodiment of the invention, cardiac arrhythmia is treated with a bead-containing agent comprising beads having a conduction-modifying property for modifying the electrical activity of the heart in a region relating to electrical activity.

In another embodiment of the invention, a heart condition is treated with a the bead-containing agent comprising a plurality of beads, each encapsulating biological material such as a cell, a gene, a peptide, a polypeptide, a protein, a neo-tissue, and any combination of one or more of the foregoing.

In another embodiment of the invention, a heart condition is treated with a multiple-component agent comprising a first component, a second component for contributing to the therapeutic effect in conjunction with the first component, and a plurality of beads dispersed in at least one of the first and second components.

In another embodiment of the invention, a heart condition is treated with a bead-containing agent comprising one or more materials having cell-recruiting and/or angiogenic-initiating properties.

In another embodiment of the invention, a heart condition is treated with a multiple-component, of which a first component comprises sodium alginate fully solubilized in an aqueous solution, a second component comprises divalent cations dispersed in solution, wherein the first and the second components interact to contribute to a therapeutic effect.

In another embodiment of the invention, a heart condition is treated with a hydrogel agent comprising alginate polymers and peptides covalently bonded to the alginate polymers.

Other embodiments of the invention include apparatus, systems, kits, and uses of or for one or more of the foregoing.

### BRIEF DESCRIPTION OF THE SEVERAL VIEWS OF THE DRAWINGS

FIG. 1 is a schematic view of a dual lumen injection procedure for beads in combination with a fibrin glue agent.

FIG. 1A is a schematic view of a single lumen injection procedure for beads.

FIG. 2A is a cross-sectional view of an illustrative region of damaged tissue associated with a cardiac structure such as along a left ventricular wall.

FIG. 2B is a schematic view of a cardiac structure delivery assembly shown during one mode of use for treating the damaged cardiac structure shown in FIG. 2A.

FIG. 2C is a schematic plan view of a therapeutic mechanical scaffolding resulting from the mode of use embodiment shown in FIG. 2B.

FIG. 3A is a schematic cross-sectional view of a biopolymer bead with an alginate core material with a covalently attached peptide moiety.

FIG. 3B is a schematic cross-sectional view of the biopolymer bead depicted in FIG. 3A with a chitosan biopolymer overcoat.

FIG. 3C is a schematic cross-sectional view of a biopolymer bead with a core material containing an alginate:peptide complex with ionically attached low molecular weight chitosan and the core surface overcoated with high molecular weight chitosan.

FIG. 4A and FIG. 4B are schematic illustration of certain aspects related to interstitial cell coupling in relation to therapeutic scaffolding.

FIG. 5 is a cross-sectional view of a heart that includes an infarcted or otherwise ischemic area of the left ventricle wall prior to treatment.

FIG. 5A is the same view of the heart shown in FIG. 5, depicting an epicardial procedure to deliver biopolymer beads to damaged cardiac tissue.

FIG. 5B is the same view of the heart shown in FIG. 5, depicting an endocardial procedure to deliver biopolymer beads to damaged cardiac tissue.

FIG. 5C shows the same view of the heart shown in FIG. 5B but after bead injection.

FIG. 6 is a cross-sectional view of a heart with a further needle injection assembly shown during use in treating an area of damaged left ventricle wall.

FIGS. 7A and B are schematic views of further respective modes of transvascular use for a cardiac structure delivery catheter to inject bead agent into a damaged area of cardiac structure such as a left ventricle wall.

FIG. 8 is a schematic view of one particular combination system for providing cardiac treatment using a multiple component bead agent.

FIG. 9 is a graph illustrating the proliferation of human umbilical vein endothelial cells in the presence of various compounds.

FIG. 10 shows the adhesion of cells to various alginates in culture.

FIG. 11 is a graph illustrating the mRNA expression from the FGF2 gene in the presence of various compounds.

FIG. 12 is a schematic view of an apparatus for generating microspheres using an electrostatic field.

FIG. 13 shows mesenchymal stem cells encapsulated in alginate beads.

### DETAILED DESCRIPTION OF THE INVENTION

The various methods, apparatus and materials described herein are suitable for use in cardiac repair, cardiac reconstruction, non-ablative conduction modification, or any combination thereof. Various polymer-based beads and hydrogels, and particularly biopolymer-based bead agents and hydrogels, may be injected into the myocardium from either inside (endocardial) or outside (epicardial) of the heart. The various biopolymer-based bead agents and hydrogels may be injected into the myocardium either alone or with other material. The various biopolymer-based bead agents and hydrogels may provide a therapeutic wall support or tissue engineering scaffold within cardiac structures of the heart, may induce angiogenesis, may recruit cells, and/or may prevent apoptosis to expedite myocardial repair/reconstruction. The biopolymer-based beads and hydrogels may contain only biopolymer material, or may further include cells, peptides, proteins, nucleic acids or other materials. The nucleic acids may be in the form of oligonucleotides, plasmids, genes or otherwise as will be recognized by those skilled in the art upon review of the present disclosure. The cells may, for example, include stem cells, fibroblasts, chondrocytes, osteocytes or other skeletal cells. The cells may be provided in the form of neo-tissues. Certain growth factors may be included either as proteins or encoded by a plasmid or gene. The biopolymer-based beads and hydrogels may particularly include fibrin factor (or fragment) E, RDG and/or RDG binding sites. Various chemo-attractants and pharmaceutical compositions as well as other therapeutically beneficial materials may also be included with the biopolymer-based beads and hydrogels. Any or all of the above as well as other materials may be included with the biopolymer-based beads and hydrogels as will be recognized by those skilled in the art upon review of the present disclosure.

A variety of biopolymers and combinations of biopolymers may be used to form the biopolymer-based beads. The biopolymers may be hydrogels. Suitable biopolymers may include fibrin glue, collagen, alginates, and chitosan for example. The biopolymer or combination of biopolymers and other material may be fabricated as beads. Various techniques may be used to limit migration or diffusion of the beads and hydrogels from the site of injection. In one technique, beads may be introduced with a biopolymer anchoring component such as fibrin glue. In another technique, beads may contain matrix-forming material such as fibrin glue encapsulated in rapidly biodegradable material. With this technique, the fibrin glue may be rapidly released from the capsule to form an in situ matrix. In another technique, beads may be provided with an adhering material at the surface for adhering to myocardial tissue. The adhering material may be formulated so that the beads are not adherent to one another within the delivery system. The beads may be coated with a suitable material so as not to interact with one another within the delivery system, or to provide a controlled-release property. Also, in certain configurations, the rate of resorption and other physical characteristics of the biopolymer system may be controlled by varying the degree of cross-linking, chemical modification and/or the molecular weight of the components using various techniques as will be recognized by those skilled in the art upon review of the present disclosure.

For example, when utilizing an alginate hydrogel as the biopolymer, the use of a low molecular weight (MW) alginate (MW˜60,000 gram/mol) as opposed to a high molecular weight alginate (MW˜120,000 gram/mol) results in a more rapid resorption regardless of whether the alginates are ionically or covalently cross-linked. See Kong, et al “Controlling rigidity and degradation of alginate hydrogels via molecular weight distribution,” Biomacromolecules, 2004, 5, 1720-1727, the disclosure of which is hereby incorporated by reference in its entirety. In certain aspects, the lifetime of scaffolds established using the biopolymer-based bead agents may be adjusted to a therapeutically beneficial duration. In another example, the certain physical characteristics may be altered by modification of the cross-linking of the alginate by changing concentrations of the divalent cation used. This may be represented by cross-linking of an alginate solution by adding 2.5 millimolar of Ca²⁺ per gram of alginate. This can result in a resulting film with a Young's Modulus of 12.3 Kilo Pascal (KPa) measured via stress-relaxation testing. By contrast, a higher spiking concentration of 62.5 millimolar of Ca²⁺ per gram of alginate may result in the resulting film having a Young's Modulus of 127 KPa. See Nicholas G. Genes et al, Archives of Biochemistry and Biophysics, 422 (2004), 161-167, the disclosure of which is hereby incorporated by reference in its entirety. To achieve desired therapeutic results when injecting into human myocardial tissue, the alginate solution may, for example, be in the range 0.1% to 2% weight/volume cross-linked alginate, wherein desirable injection volumes may be in the range of approximately 0.1 to 1.5 milliliters. In this range, the cross-linking of the alginate solutions may be accomplished with addition of divalent cations such as Mg²⁺, Sr²⁺, or Ba²⁺. In other embodiments, chitosan may be use in cross-linking alginate solutions. See U.S. Pat. No. 6,165,503 issued Dec. 26, 2000 to Gaserod, the disclosure of which is hereby incorporated by reference.

Among other subject matter, described herein are novel systems and methods, which may include novel compositions of matter, which advantageously are effective for: treating of ischemic myocardium, such as that associated with myocardial infarction; supporting of damaged cardiac structures, such as infarcted regions of ventricles in the heart; modifying electrical conduction within cardiac structures; reversing negative left ventricular wall remodeling; treating cardiac conditions following myocardial infarction; treating ischemic cardiac tissue structures; treating infarcts; treating cardiac conditions associated with congestive heart failure; and treating cardiac conditions associated with dilated cardiomyopathies and in more specific examples conditions associated with congestive heart failure or acute myocardial infarction such as for example ischemic tissue or infarcts.

Some of these systems and methods, which may include novel compositions of matter, may involve: a scaffold within cardiac tissue structures for enhanced retention and viability of implanted cells within cardiac tissue structures; an injectable scaffolding agent for injection into cardiac structures; injection of therapeutic, internal wall scaffolding within cardiac structures; and/or therapeutic mechanical scaffolding within a cardiac structure as an internal wall support.

Other of these systems and methods, which may include novel compositions of matter, may involve: therapeutic angiogenesis to transplanted cells within a patient; angiogenesis into cardiac tissue structures, including those receiving cell implant therapy, such as within infarcted ventricle walls; inducement or enhancement of therapeutic angiogenesis in cardiac structures or in injected cardiac structure scaffolds; and/or inducement of angiogenesis in a cardiac structure at least in part with an injected polymer agent.

Other of these systems and methods, which may include novel compositions of matter, may involve: enhanced retention of transplanted cells in a patient; enhanced retention and viability of implanted cells within cardiac tissue structures; retention of living cells in a therapeutic mechanical scaffolding within a cardiac structure by use of an injectable combination of such living cells with a polymer agent; enhanced deposition of cells into a cardiac structure of a patient; and/or an induced deposition of autologous cells within a cardiac structure of the patient at least in part with an injected polymer agent.

Other of these systems and methods, which may include novel compositions of matter, may involve: additional cellular recruitment and deposition into cardiac tissue structures receiving cell implant therapy; and/or use of factors adapted to recruit endogenous cells, including providing a cellular deposition recruiting factor.

Other of these systems and methods, which may include novel compositions of matter, may involve: modifying conduction in various areas of the heart by injection of material; reversibly blocking conduction in certain areas of the myocardium to treat cardiac arrhythmias; and/or reversibly reestablishing conduction in certain areas of the myocardium to treat cardiac arrhythmias.

It is to be appreciated that these systems and methods may be used individually or in various combinations with one another, and may involve more detailed aspects which may also be beneficial with respect to achieving the technological and other effects of one or more of the preceding aspects, or otherwise providing other substantial benefits.

The various methods and apparatus described herein, which may include various compositions of matter that can advantageously hinder and, in some embodiments, can reverse the negative remodeling process of infarct related wall thinning and aneurysm formation. Accordingly, aspects of the present inventions may provide a treatment for Congestive heart failure by the prevention and reversal of left ventricular aneurysms and improved left ventricular function. Further, aspects of the present inventions may provide a treatment for chronic ischemic cardiomyopathy and idiopathic dilated cardiomyopathy by increasing or otherwise improving wall thickness.

Reference is made herein to providing scaffolding in hearts, generally sufficient to achieve therapeutic result to damaged cardiac tissue. It is to be appreciated that such terms as “support” and “scaffold” are intended to mean, in one regard, that a significant result of the intervention is providing a mechanically relevant, structural change to the tissues of the heart, which may be with regard to one structural aspect or several. The structural change may be of varying degrees, ranging from rigid to compliant, and may be achieved by various mechanisms, including matrices as well as unlinked particles imbedded in interstitial regions of the myocardium. In a similar regard, at some level it may be the case that most materials have some injectability and some scaffolding features to many if not most types of tissues. However, a material is herein considered substantially an injectable scaffolding material with respect to cardiac tissues if such material causes measurable benefit, and furthermore in most circumstances that is not outweighed by more deleterious detriment. Moreover, it is also contemplated that while chronically improved support to damaged cardiac tissue has been observed, such chronic results may not be required to gain value and benefit from treatment in all cases

The biopolymer-based bead agents, chitosan hydrogel-based agents, alginate hydrogel-based agents, and other agents such as those described in U.S. Patent Application Publication No. 2005/0271631 published Dec. 8, 2005 to Randall J. Lee et al. (“Lee et al. application), which is incorporated by reference in its entirety, may be injected from within the heart as described in the Lee et al. publication, or from outside of the heart in the manner described below. Some exemplary suitable biopolymers for injection, beads and hydrogels include fibrin glue, collagen, alginates, and chitosan. In addition to biopolymers, various biocompatible polymers may also be used for injection and/or bead formation. Such biocompatible polymers may include various polymers that can be tolerated by the body and may be delivered into the myocardium in accordance with the disclosed methods. In certain aspects, the polymer utilized may be in the form of a hydrogel. In other aspects, the polymer may be in the form of a bead or a bead core. In other aspects, the bead or injected material may be a mixture of materials. Other suitable polymers include cyanoacrylate glues. Other suitable polymers include polyethylene oxide (“PEO”), polyethylene oxide-poly-l-lactic acid (“PLLA-PEO block copolymer”), poly(N-isopropylacrylamide-co-acrylic acid) (“poly(NIPAAm-co-Aac)”), a pluronic agent, and poly-(N-vinyl-2-pyrrolidone) (“PVP”), polyethylene glycol (“PEG”), polyvinyl alcohol (“PVA”), hyaluronic acid, sodium hyaluronate, and other polymers other formulations that may be injectable and/or may be formed into beads and/or hydrogels as will be recognized by those skilled in the art upon review of the present disclosure.

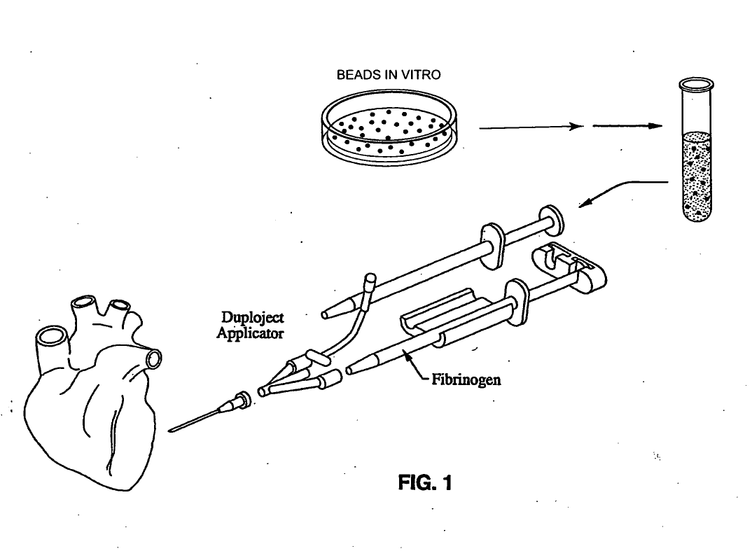

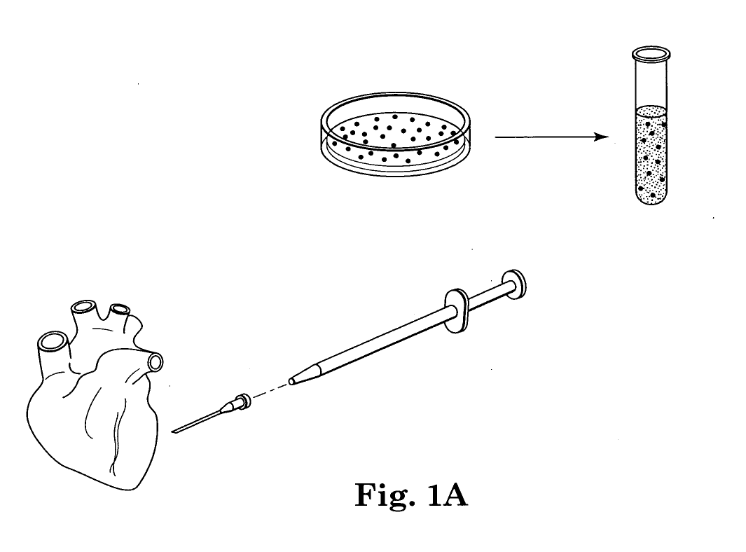

Single injections of agent with a single lumen catheter such as shown in FIG. 1A is suitable for agents that are designed not to clog a single lumen, because of the speed of injection, lessening of trauma, and relative ease of injection. As illustrated, the catheter is in the form of a syringe having a plunger to advance the material into the patient. The syringe includes a needle in communication with the passage within the syringe. The needle is generally configured to penetrate the myocardial tissue to permit material to be deposited at a desired position within the myocardium. However, a multiple-lumen catheter such as shown in FIG. 1 may be used if desired to deliver a multiple-part agent, an agent and an initiator, or other such multiple-part formulation. As illustrated, the catheter is in the form of a two barreled syringe having a first plunger to advance a first material through a first passage and a second plunger to advance a second material through the second passage. As illustrated for exemplary purposes, the multiple-lumen catheter is configured to intermix the first and the second material before introducing the mixed materials into the patient. The syringe includes a single needle in communication with both the first passage and the second passage within the syringe. The needle is generally configured to penetrate the myocardial tissue to permit material to be deposited at a desired position within the myocardium. In the illustrated and various alternative embodiments, the parts of a multiple-part formulation may be provided contemporaneously or serially, depending on the properties of the formulation. Multiple single lumen catheters may be used if desired. The formulation and catheter or catheters may be provided in kit form, or as individual components of an injection system.

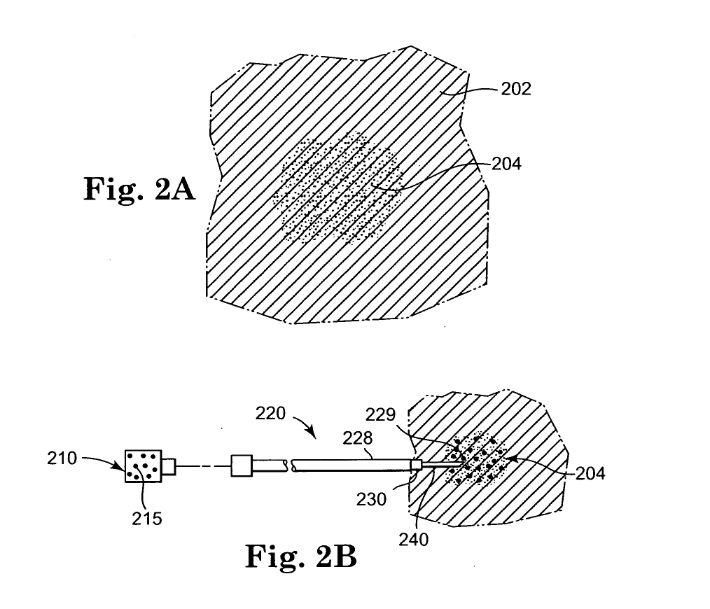

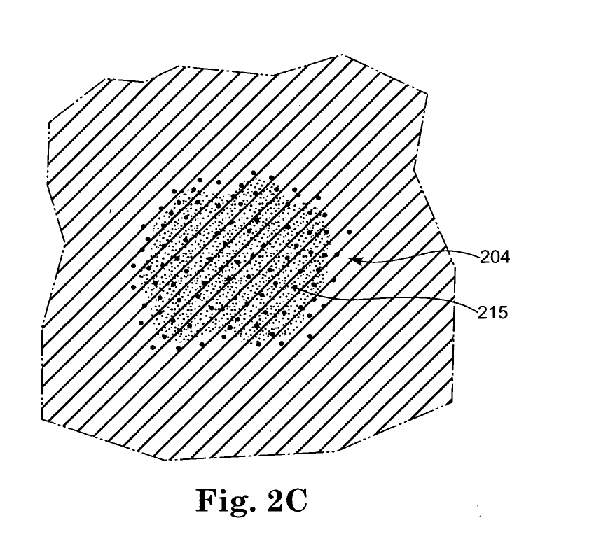

The site of injection may be controlled in the following manner. FIG. 2A schematically shows a region of cardiac tissue 202 along a ventricle that includes an infarct region 204 or otherwise ischemic region of myocardium. As shown in FIG. 2B, the distal end portion 228 of a catheter 220, which may be a single lumen catheter or a multiple lumen catheter, is delivered to the region at a location associated with the region 204 such that the desired material 215 may be injected into that zone 204. This is done for example using a mapping electrode 230 provided at distal needle tip 229 and via an external mapping/monitoring system coupled to proximal end portion of catheter 220 outside of the body. Needle 240 is punctured into the tissue at the location, and is used to inject the desired material 215 from source 210, also coupled to proximal end portion of catheter 220 outside of the body. According to this highly localized injection of the material 215 into the location of the infarct, the ventricular wall at that location is supported by the desired molecular scaffold within the tissue structure itself. According to further aspects and embodiments herein described, cellular scaffolding may also be thus provided, angiogenesis of the area may thus be created, and negative remodeling may be prevented, inhibiting progression and possible reversal of harmful cardiomyopathy. An illustrative scaffolding result is illustrated in FIG. 2C.

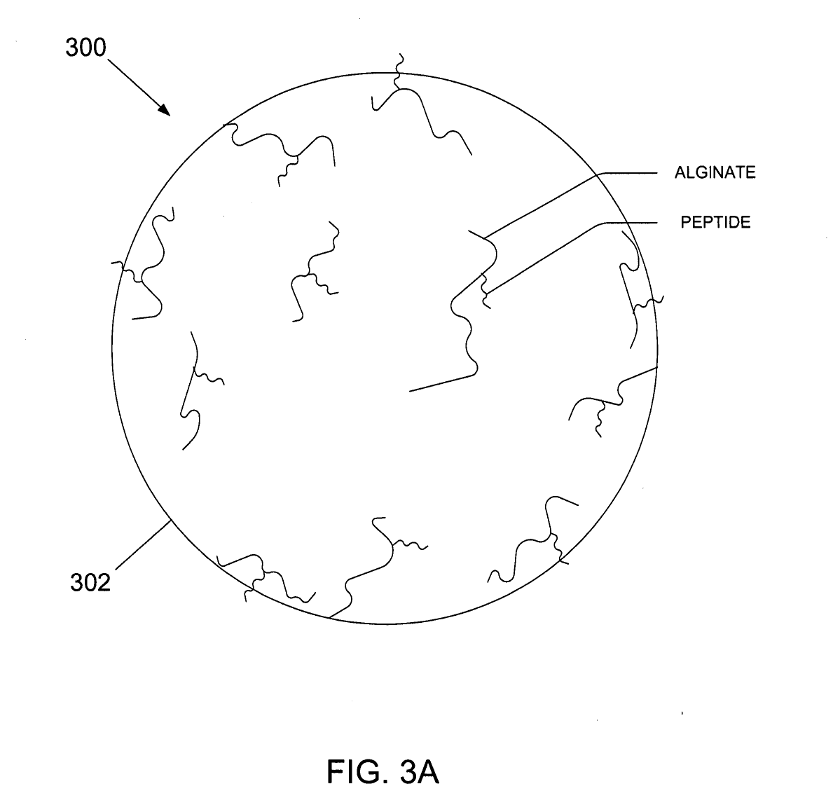

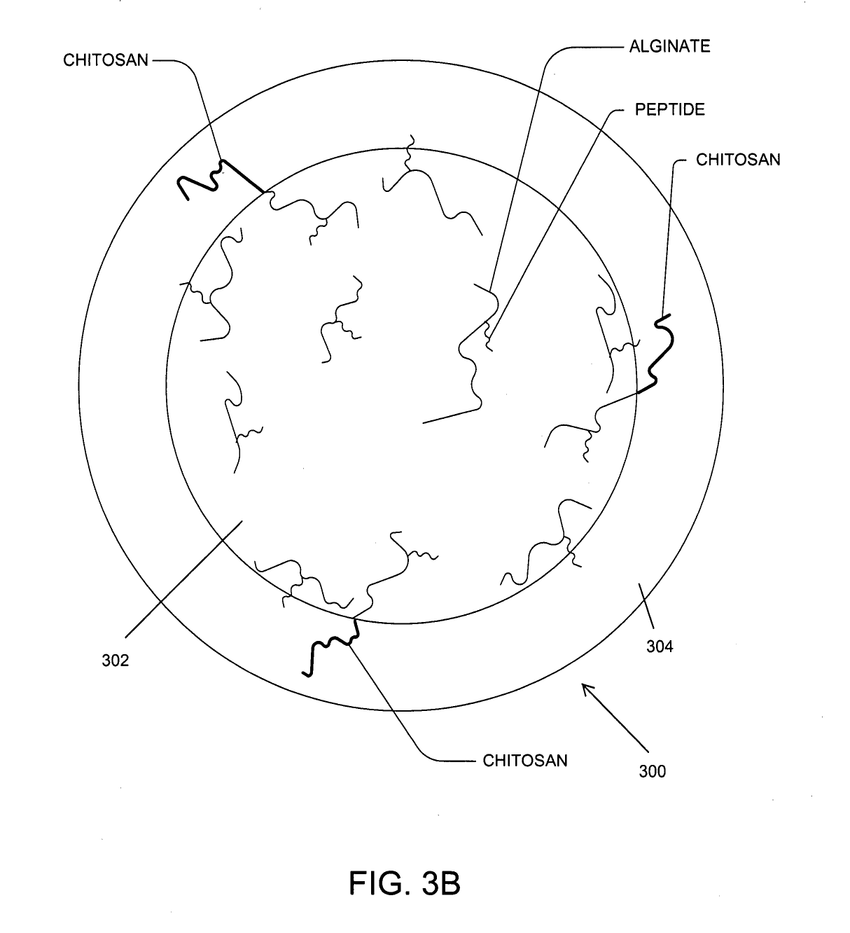

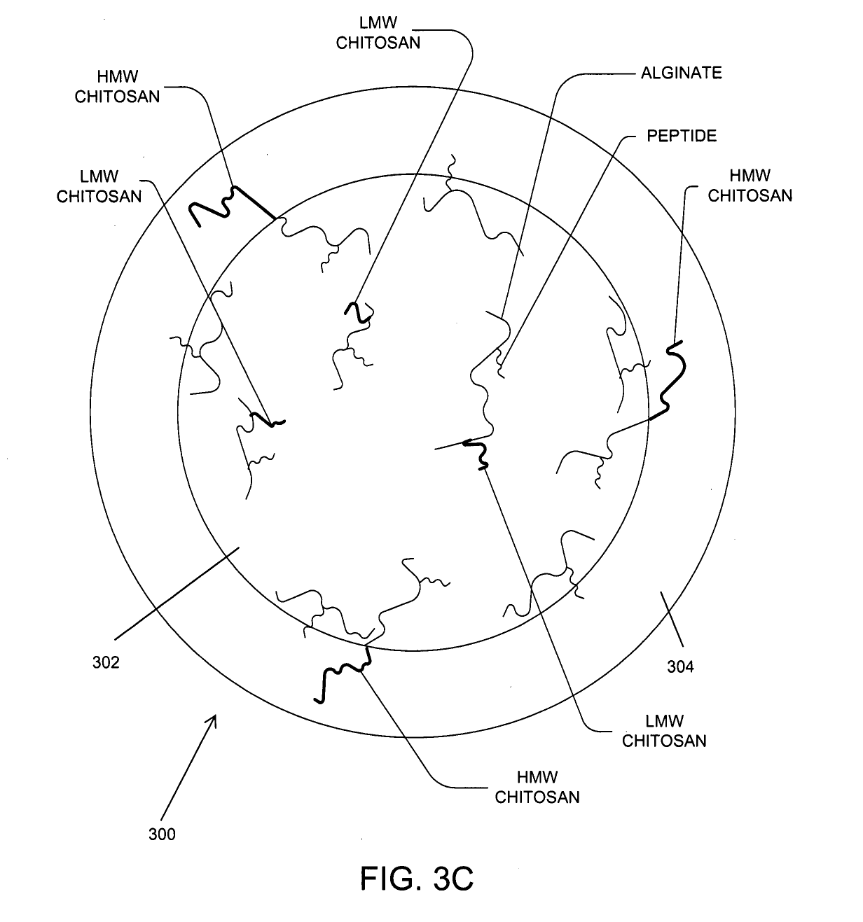

A cross-sectional schematic representation of a biopolymer bead 300 is shown in FIG. 3A. The bead 300 may have a geometrical core 302 of alginate type material. The bead core's 302 surface geometry may be spherical, elliptical, out of round, and/or contain surface irregularities. The term bead as used herein is intended to encompass all of the aforementioned geometries.

The bead core 302 may, if desired, have peptides moieties covalently bonded to the alginate polymer. Suitable peptides include, but are not limited to, the polypeptides: arginine-glycine-aspartic acid (RGD), glycine-arginine-aspartic acid-valine-tyrosine (GREDVY), glycine-arginine-glycine-aspartic acid-tyrosine (GRGDY), glycine-arginine-glycine-aspartic acid-serine-proline (GRGDSP), tyrosine-isoleucine-glycine-serine-arginine (YIGSR), valine-alanine-proline-glycine (VAPG), and arginine-glutamic acid-aspartic acid-valine (REDV). In addition, various growth factors may be bonded to the alginate polymer, including but not limited to, EGF, VEGF, b-FGF, FGF, TGF, and TGF-β. Various other compounds including proteoglycans among others may also be bonded to the alginate polymer. These and additional peptides may be synthesized using various techniques or otherwise obtained as will be recognized by those skilled in the art.

A variety of techniques may be utilized to couple peptides to the alginate polymer backbones. These methods include various synthetic methods which are in general known to those of ordinary skill in the art. Some conventionally known methods for attachment or immobilization of adhesion ligands may be used include those found in U.S. Pat. No. 6,642,363 issued Nov. 4, 2003 to Mooney et al., the disclosure of which is hereby incorporated by reference in its entirety.

For example, certain methods may form an amide bond between the carboxylic acid groups on the alginate chain and amine groups of the peptides. Other useful bonding chemistries may include the use of carbodiimide couplers, such as 1,3-Dicyclohexylcarbodiimide (DCC) and N,N-diisopropyl-carbodiimide (DIC—Woodward's Reagent K). Since the peptides contain a terminal amine group for such bonding. The amide bond formation may also be catalyzed by 1-ethyl-3-(3-dimethylaminopropyl) carbodiimide (EDC), which is a water soluble enzyme commonly used in peptide synthesis. EDC reacts with carboxylate moieties on the alginate backbone creating activated esters which are reactive towards amines. R—NH₂ represents any molecule with a free amine (i.e. lysine or any peptide sequence N-terminus). To reduce unfavorable side reactions, EDC may be used in conjunction with N-hydroxysuccinimide, N-hydroxysulfylsuccinimide or 1-hydroxybenzotriazole (HOBT) to facilitate amide bonding over competing reactions.

The reaction conditions for this coupling chemistry can be optimized, for example, by variation of the reaction buffer, pH, EDC:uronic acid ratio, to achieve efficiencies of peptide incorporation between 65 and 75%, for example. Preferably, the pH is about 6.5 to 7.5. The ionic concentration providing the buffer (e.g. from NaCl) is preferably about 0.1 to 0.6 molar. The EDC:uronic acid groups molar ratio is preferably from 1:50 to 20:50. When HOBT is used, the preferred molar ratio of EDC:HOBT:uronic acid is about 4:1:4. Both surface coupling, as well as bulk coupling of alginate can be readily obtained with this exemplary coupling chemistry. Therefore, by manipulation of surface and bulk coupling, materials having one type of molecule coupled internally in the matrix and another type of molecule coupled on the surface can be provided, for example. In addition to having peptides dispersed throughout the core region of the bead, it may be advantageous to have specific cell attachment peptides (for example RGD and/or GREDVY) exposed on the surface of the bead and in sufficient concentration to enhance anchoring to underling endothelial tissue. To increase the surface concentration of cell attachment peptides, the beads may be dip coated or spray coated with a solution/mist containing the peptide chemistry to ensure all available potential alginate bonding sites on the surface are saturated with cell attachment peptides.

The following two experiments conjugate the GRGDY pentapeptide to the alginate polymer backbone through the terminal amine of the peptide:

### EXAMPLE 1

In the first example, alginate was modified with the GRGDY peptide in solution to create a homogeneously modified material. The chemistry was optimized for a peptide density of 1 mg GRGDY per gram alginate as it is 2.5 orders of magnitude greater than the minimal RGD ligand spacing determined necessary for cell attachment when extrapolated to three-dimensional space (calculations based on a body centered cubic unit cell). Alginate chemistry was performed in 1% (v/v) alginate solutions in 0.1 M MES buffer at varying pH (6.0-7.5) and NaCl concentrations (0.0-0.7 M) for 12 or 20 hours. Sulfo-NHS was dissolved in the alginate solution at a ratio of 1:2 to EDC, and EDC was next added as a percentage of uronic acids available for reaction (0-50%). The GRGDY peptide was added after 5 min with 125I-GRGDY as a tracer molecule (activities of 2-5 μCi per reaction). The alginate product was purified by dialysis (3500 MWCO) against ddH2O for four days and lyophilized until dried. The resultant solid was weighed and dissolved in ddH2O to obtain a 0.5% (w/v) solution, of which the activity of 0.5 ml samples (three per condition) were counted with a Packard-Bell Gamma Counter. The activity, in counts per minute, were compared to the initial reaction solution activities, and reaction efficiencies were calculated taking into account the 125I decay. A range of ligand densities in the bulk was produced by using optimized chemistry and changing the GRGDY concentrations in the reactions.

### EXAMPLE 2

In the second example, pre-formed hydrogels were modified with the GRGDY peptide using similar chemistries. Calcium cross-linked alginate hydrogels were prepared from 2% (v/v) alginate solutions in ddH2O containing 0.2% (w/v) Na(PO₄)₆ (Alfa, Ward Hill, Mass.). Calcium sulfate was added to alginate in 50 ml centrifuge tubes as a water-based slurry at 0.41 g CaSO₄/ml ddH₂O, with 0.2 ml of the slurry added for every 5 ml of the 2% alginate solution to be gelled. The gelling solution was shaken rapidly and cast between parallel glass plates with 2 mm spacers to prepare gel films. Hydrogel disks were punched out of the film with a hole-punch (McMaster-Carr, Chicago, Ill.) for modification of the hydrogel. The hydrogel disks were derivatized with RGD using un-buffered EDC chemistry in ddH2O with sulfo-NHS as the co-reactant. Sulfo-NHS and EDC were added to 40 ml ddH2O at the same ratios as modification Example 1, followed by addition of the GRGDY peptide. Example 2 reactions were performed in 50 ml centrifuge tubes on 10-12 hydrogel disks at a time for 20 h. Surface densities of GRGDY were estimated with this method assuming a 50 nm penetration of reactants, since uronic acid available for reaction greatly outnumbered the molar quantities of reactive species. Peptide surface densities were quantified with the 125I-GRGDY tracer molecule as described above. Although hydrogel discs were utilized in the preceding example, those skilled in the art would recognize the application of the present methodologies to alginate bead cores.

The bead core 302 may be manufactured using various devices and techniques that will be recognized by those skilled in the art upon review of the present disclosure. These devices and techniques may utilize laminar jet break-up, high voltage driven, and coaxial-air-driven technologies as well as other technologies to produce a bead core of appropriate size and shape. One such technique is electrostatic bead generation, which is particularly suitable for manufacturing beads as small as about 200 μm. In this technique, a solution containing dissolved alginate material is injected into a needle oriented vertical, aimed downward. Directly below the needle tip, displaced a predetermined distance (the dropping distance) is placed a capturing aqueous solution. An electrostatic potential of typically a few kilovolts is applied between the needle tip and the capturing aqueous solution to pull the droplets from the needle tip. The individual droplets are then harvested one-by-one as they fall into the capturing aqueous solution. The size of the beads can be controlled by varying any of the following variables: the inside diameter of the needle tip, the magnitude of the electrostatic potential, the concentration of alginate in solution, the dropping distance, and combinations thereof. Also, the alginate core material may, or may not, have a peptide moiety covalently attached to the alginate biopolymer, as explained above, prior to bead fabrication.

For some medical applications, the bead 300 outlined above may include a bead core 302 with or without a covalently, ionically or otherwise attached moieties. These may include, for example, peptides, chitosan, poly-lysine and other moieties that will be recognized by those skilled in the art and are disclosed in the present application. When alginate is used for the bead core 302, the alginate formulations can have certain angiogenic properties and certain identified peptides have been known to have cell signaling properties, i.e., attracting stem cells amongst other cellular types to the area of injection.

In applications where it may be desired to anchor the bead(s) 300 at site of injection, it may be desirable to overcoat the bead core 302 with a coating 304. The coating 304 may be adhesive. In one aspect, the coating material may be attached to the both the alginate surface on the inner surface of the coating 304 and to myocardial tissue on the outer surface of the coating 304. The coating 304 may be chemically bonded and/or mechanically secured to the bead core 302 to form bead 300. Given that both the alginate and the myocardial tissue have negative bonding sites available, a coating 304 with a positive charge density may be appropriate.

Chitosan is one exemplary coating 304 with a positive charge density. Chitosan and its derivatives are biopolymer materials used in a wide range of medical applications. Chitosan is a linear polysaccharide, and given its positive charge density is a bioadhesive which readily binds to negatively charged surfaces such as mucosal membranes. FIG. 3B is a schematic representation of bead 300 having a bead core 302 with a coating 304. As shown, the bead core 302 is comprised of at least an alginate and the coating 304 is composed of at least a chitosan. The alginate bead core 302 may be manufactured by the technique describe above or by any known equivalent to those skilled in the art of micro-encapsulation. The chitosan coating 302 may be applied by dip coating or other known procedures, wherein the chitosan may ionically bond to the available negative sites on the alginate surface. Given this, the chitosan may act as an anchor to immobilize the beads 300 to the negatively charged myocardial tissue. This may provide temporary mechanical integrity to tissue damaged by a myocardial infarction. As used in this sense, the chitosan overcoat material is temporary in that it will eventually be enzymatically dissolved. Accordingly, “anchoring time” may be prolonged by increasing the thickness of the chitosan overcoat.

An alternative approach to increasing the “anchoring time” without relying solely on increasing the thickness chitosan coating 304 is depicted in FIG. 3C. An alginate bead core 302, with or without covalently attached peptides. The alginate bead core 302 may then be dip coated in a solution containing a mixture of both low and high molecular weight chitosan derivatives. The low molecular weight chitosan derivatives may be sufficiently small and have sufficient kinetic energy to diffuse into the bead core 302 and, in some cases, ionically bond with alginate in the bead core 302. Upon completion of the dip coat, the now alginate:chitosan impregnated bead core 302 may have an overcoat consisting of a mixture of both high and low molecular weight chitosan. However, when now dissolved down to and into the bead core 302, there may be a sufficient population of chitosan polymers (ionically bonded to alginates in the core) and with sufficient positive charge sites left available to prolong the anchoring process while the bead core 302 itself is biodegrading away.

Since manufacturing techniques such as the electrostatic technique among other techniques are capable of making very large beads on the order of a few millimeters, the upper bead size limit depends on a number of practical factors other than the manufacturing technique. Bead sizes in excess of 500 μm and with good myocardial adhesion properties may be suitable for direct injection into damaged myocardial tissue, provided the beads do not encapsulate living cells. However, if living cells are to be encapsulated, the upper size limit may be dictated by diffusion limitations of nutrients such as oxygen for the encapsulated cells, with beads on the order of 500 μm or less being typical. For the alginate and/or chitosan encapsulation of cells, proteins, or other biological materials using known bead generation techniques, for example, an appropriate size range of the beads for direct injection into damaged myocardial tissue is from about 30 μm to about 500 μm.

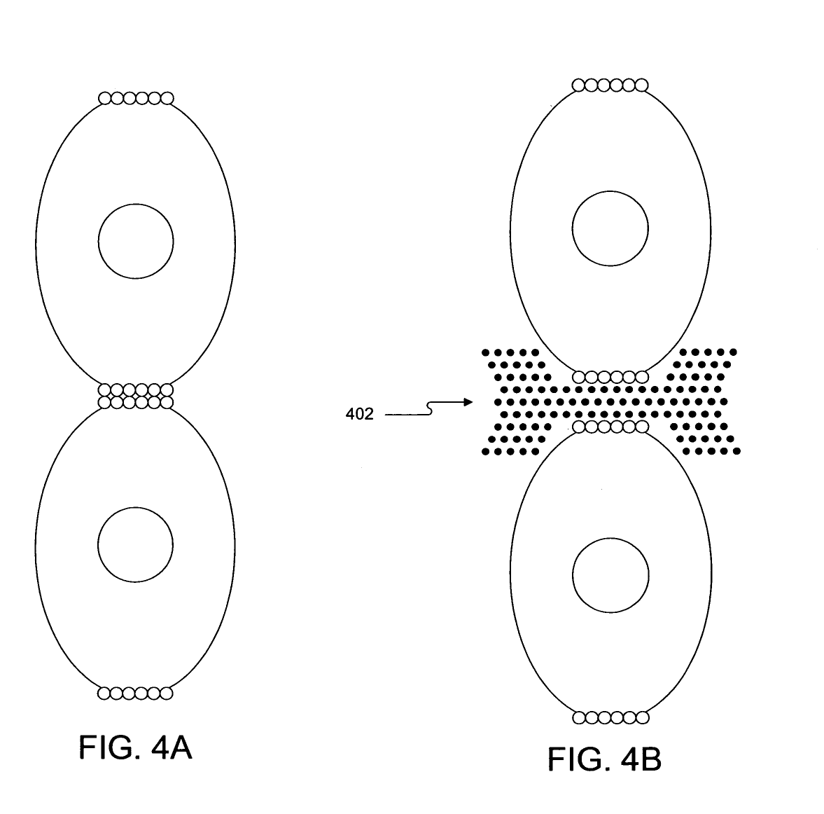

In addition to the mechanisms of action elsewhere herein described, the injected material may also alter the electrical characteristics of the location into which it is injected. Where the injected material contains a generally non-conductive biopolymer, its deposition in the artificial extracellular scaffold of tissues of the heart may result in physical separation of cells in the region of injection. FIGS. 4A and 4B show transition between a cellular matrix in an initial gap junction condition having separation “d”, as shown in FIG. 4A, and in a post-treatment condition wherein the spacing between cells is physically separated to a larger separated distance “D”, as shown in FIG. 4B. These separations may be sufficient to raise the action potential to stimulate conduction between cells to such level that conduction is blocked or otherwise retarded sufficiently to potentially result in arrhythmia.

Where conduction is desired along the scaffold region, conductive additives in the artificial extracellular scaffold may be added, or gap junction enhancement may be otherwise achieved such as by supporting cells modified for overexpression of Connexin 43 (Cx43) protein. When the scaffold is configured as beads, the cells may be in the form of skeletal muscle cells genetically modified to overproduce Cx43. The cells may be encapsulated in the beads and introduced into the myocardium. It is contemplated that such embodiments of the scaffold may incorporate, for example, cells and related gap-junction enhancing materials, and utilize various related methods, similar to those described in U. S. Patent Application Publication No. US 2003/0104568 published Jun. 5, 2003 (Lee, Methods and compositions for correction of cardiac conduction disturbances), and PCT Patent Application Publication No. WO 03/039344 published May 15, 2003 (Lee, Methods and compositions for correction of cardiac conduction disturbances), the disclosures of which are hereby incorporated by reference in their entireties.

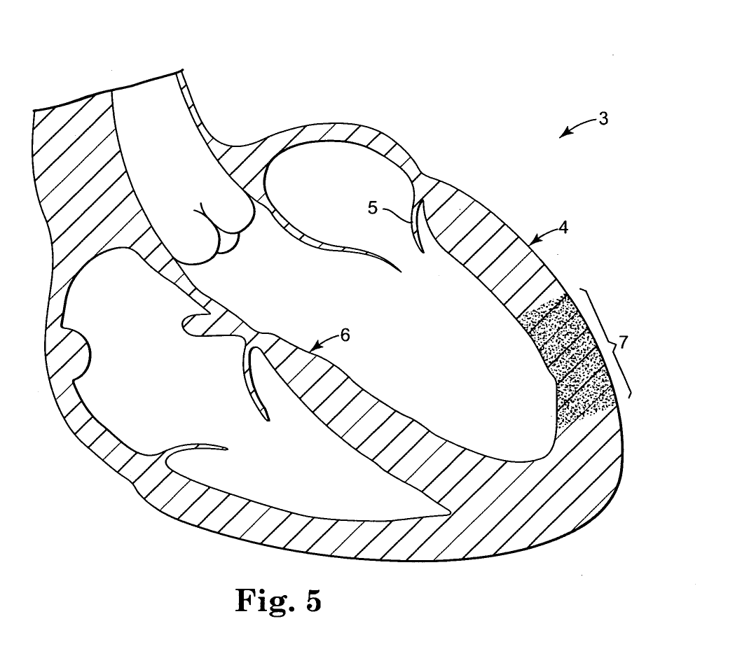

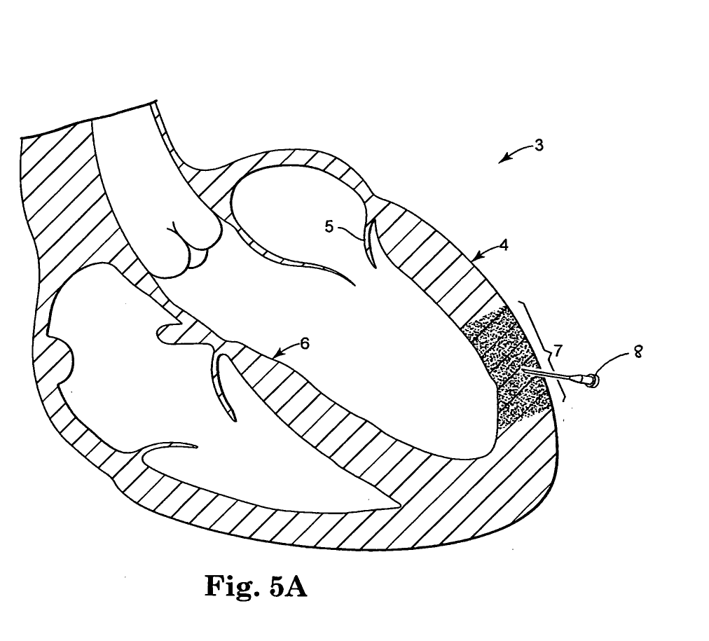

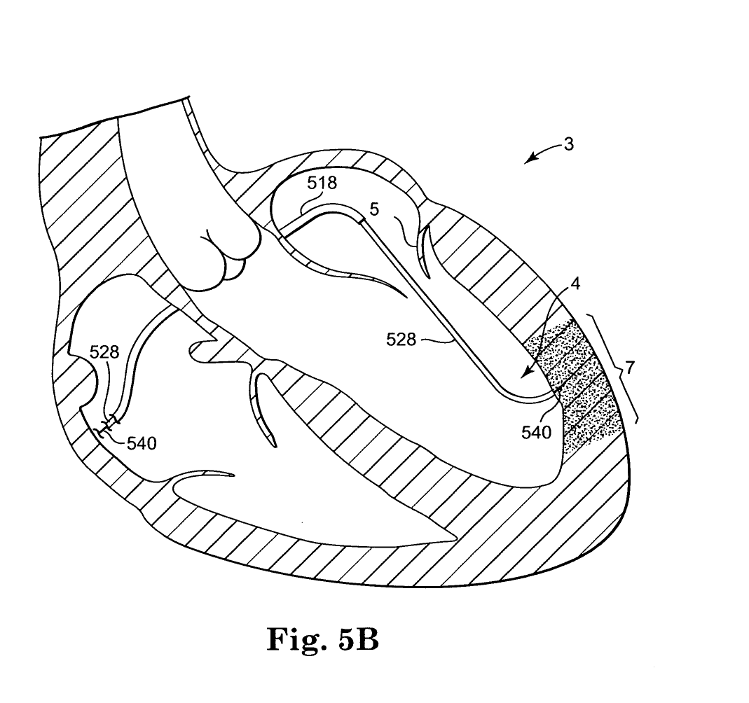

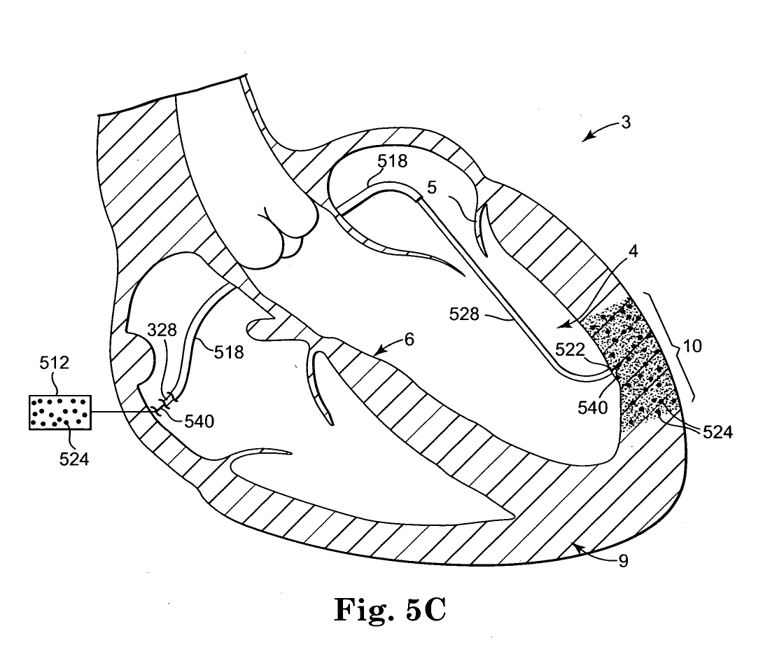

Various modes of treatment may be applied to an infarcted heart. FIG. 5 shows an example of an infarcted heart that includes left ventricle 4, mitral valve 5, inter-ventricular septum 6, and an infarct zone 7. The infarcted region 7 of the left ventricle 4 is shown prior to treatment.

FIG. 5A shows the distal end 8 of a delivery system shown embedded in infarct zone 7, which may deliver biopolymer beads to the damaged cardiac tissue. In one mode of delivery, the distal end 8 may be a needle inserted epicardially during open chest surgery. Alternatively, the needle may be inserted endocardially (not shown). In a minimally invasive mode of delivery, a catheter is inserted percutaneously and routed proximal to the infarct zone 7. The minimally invasive surgical procedure may involve guiding the catheter to the infarct zone 7 utilizing laparoscopic surgical techniques or other imaging modalities. Once guided proximate the infarct zone, the laparoscopic surgeon may have multiple options as to delivering the biopolymer beads indwelling to the infarct zone 7. In one scenario, an injection needle housed, and protected, within the distal end of the catheter while enroute to the infarct zone 7 may be caused by surgeon initiation to protrude a preset linear dimension into the infarct zone 7. In another embodiment, the needle may be mechanically preset to protrude in a series of digital microsteps. The needle may then be retracted into the “home” position within the catheter, the catheter guided to an adjacent location and the procedure repeated as many times as deemed medically necessary. In another embodiment, the “needle-less embodiment”, the delivery catheter may be configured with a miniature air-gun apparatus near the distal tip of the delivery catheter which may imbed controlled dosages of beads via aerosol bombardment. The air-gun delivery apparatus may be programmed to increase the nozzle velocity in digital increments during aerosol bursts, to deliver beads in varying depth achieving similar results above in having the needle protrude digital microsteps.

FIG. 5B shows an agent delivery system that includes a percutaneous epicardial delivery catheter 518 slideably engaged over an agent delivery catheter 528 that is further slideably engaged over a delivery needle assembly 540. Agent delivery catheter 528 is delivered into the left ventricle 4 by manipulating its proximal end portion (not shown) externally of the body via a percutaneous approach either through the femoral artery or alternate entry site, and is advanced into the left ventricle 4 via delivery catheter 518. The distal tip 522 of the delivery catheter 528 is positioned within the left ventricle 4 against the wall where infarct zone 7 is identified.

As shown in FIG. 5C, a source of agent 512 is coupled to a proximal end portion of the delivery catheter. A volume of the agent 524 from the source is then delivered through a delivery lumen (not shown) within the agent delivery catheter 528 and into infarct region 7. This may be accomplished using pressure alone, though in certain beneficial embodiments a needle tip 540, which may in fact either integral with the delivery catheter or slideably disposed therein, is used to inject the agent 524 into the tissue. Where such a separate cooperating needle is used, the internal bore of the needle will be coupled proximally with the source of agent.

It is to be appreciated according to the embodiments herein described that one or more (e.g. an array) of electrode members may be delivered subsequent to, before, or simultaneous with delivery of agent 524 for enhancing conduction of the scaffolded region, or for mapping purposes to locate the proper injection site and pattern or area.

The depth of injection via needle delivery may be controlled by standard surgical techniques well known to those skilled in the art of cardiac surgery.

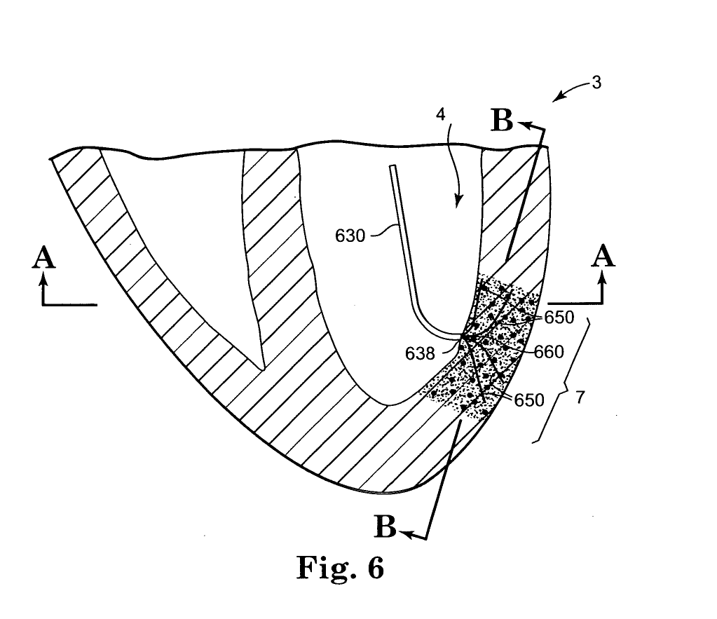

Many other techniques may be used to introduce the bead agent. An illustrative arrayed scaffolding injection assembly is shown in FIG. 6. The array of injection members 650 is shown in angular arrangement within a transversely cross-sectioned heart for illustration, but they may share a planar orientation, such as in a plane transverse to the plane of cross-section shown for heart 3. Accordingly, anchor element 660 is located within a region of septal wall tissue that is bound by injection members 650 that have been positioned at unique respective locations around such central anchor 660 across the region. By providing scaffolding injection members 650, central injection member 660, and tip 638 as a recording electrode, the tissue bounded by injection members 650 may be substantially supported with injectate, such as for treating infarct, congestive heart failure, or cardiomyopathy.

It is to be appreciated that while needle or “end-hole” injection delivery catheters may be used to inject the agent, more complex “needle” injection devices are herein contemplated, such as for example using screw needles with multiple ports along the screw shank, or in another example needle devices with multiple adjacent needles. Multiple needles may be employed in a spaced fashion over a region for delivery, allowing for the injection and subsequent diffusion or other transport mechanisms in the tissue to close the gaps between scaffolds from discrete injection sites and cover the region as one example of an equivalent approach to continuous, uninterrupted contact of a delivery member over that region. It is also to be appreciated that other delivery systems including the system shown in FIG. 6 may be beneficially provided along a larger region of tissue generally achievable by traditional “end-hole” injection approaches. More specifically, the agent may be injected along a substantial portion of a ventricle wall, both wide and deep.

Generally, it is desired to match delivery of cells and other scaffolding closely to the damaged area, so that the delivery catheter desired to achieve a dispersed injection would be suitably adapted to inject the scaffolding material along a predetermined expansive and shaped region. Such custom delivery and resulting scaffolding provides for reliable and controlled impact of the therapy. In other words, “contacting” a region of tissue is considered contextual to the particular embodiment or application, and may be substantially continuous and uninterrupted contact in certain circumstances, or in others may have interruptions that are considered insignificant in the context of the anatomy or more general use.

For the purpose of further illustration, other more specific examples of delivery devices and methods that may be modified according to this disclosure are variously disclosed in one or more of the following documents: U.S. Pat. No. 5,722,403 issued Mar. 3, 1998 to McGee et al.; U.S. Pat. No. 5,797,903 issued Aug. 25, 1998 to Swanson et al.; U.S. Pat. No. 5,885,278 issued Mar. 23, 1999 to Fleishman; U.S. Pat. No. 5,938,660 issued Aug. 17, 1999 to Swartz et al.; U.S. Pat. No. 5,971,983 issued Oct. 26, 1999 to Lesh; U.S. Pat. No. 6,012,457 issued Jan. 11, 2000 to Lesh; U.S. Pat. No. 6,024,740 issued Feb. 15, 2000 to Lesh et al.; U.S. Pat. No. 6,071,279 issued Jun. 6, 2000 to Whayne et al.; U.S. Pat. No. 6,117,101 issued Sep. 12, 2000 to Diederich et al.; U.S. Pat. No. 6,164,283 issued Dec. 26, 2000 to Lesh; U.S. Pat. No. 6,214,002 issued Apr. 10, 2001 to Fleischman et al.; U.S. Pat. No. 6,241,754 issued Jun. 5, 2001 to Swanson et al.; U.S. Pat. No. 6,245,064 issued Jun. 12, 2001 to Lesh et al.; U.S. Pat. No. 6,254,599 issued Jul. 3, 2001 to Lesh et al.; U.S. Pat. No. 6,305,378 issued Oct. 23, 2001 to Lesh; U.S. Pat. No. 6,371,955 issued Apr. 16, 2002 to Foeman et al.; U.S. Pat. No. 6,383,151 issued May 7, 2002 to Diederich et al.; U.S. Pat. No. 6,416,511 issued Jul. 9, 2002 to Lesh et al.; U.S. Pat. No. 6,471,697 issued Oct. 29, 2002 to Lesh; U.S. Pat. No. 6,500,174 issued Dec. 31, 2002 to Maguire et al.; U.S. Pat. No. 6,502,576 issued Jan. 7, 2003 to Lesh; U.S. Pat. No. 6,514,249 issued Feb. 4, 2003 to Maguire et al.; U.S. Pat. No. 6,522,930 issued Feb. 18, 2003 to Schaer et al.; U.S. Pat. No. 6,527,769 to Langberg et al.; U.S. Pat. No. 6,547,788 to Maguire et al.; and US Patent Application Publication No. 2005/0271631 published Dec. 8, 2005 in the name of Lee et al., all of which are hereby incorporated by reference in their entirety. To the extent that these references variously relate to ablating tissue or other therapeutic uses than cell or polymer scaffolding delivery or treating the conditions contemplated hereunder, certain aspects of the respective catheter systems and therapy may be modified or otherwise per the intent and objects of this disclosure as appropriate to one of ordinary skill. For example, where ablation devices are disclosed, various related elements such as ablation electrodes, leads, transducers, optical assemblies, or the like, would be replaced with suitable elements for injecting the scaffolding materials of the type described herein. Other related elements such as ablation actuators, e. g. power sources, would be replaced with suitable sources of injectable material, and luminal structures of the delivery assemblies may be also suitably modified to provide for such injection to replace the prior modes of coupling such as electrical leads, etc. Moreover, certain aspects such as mapping and monitoring arrays and assemblies and methods maybe combined with the various features described herein.

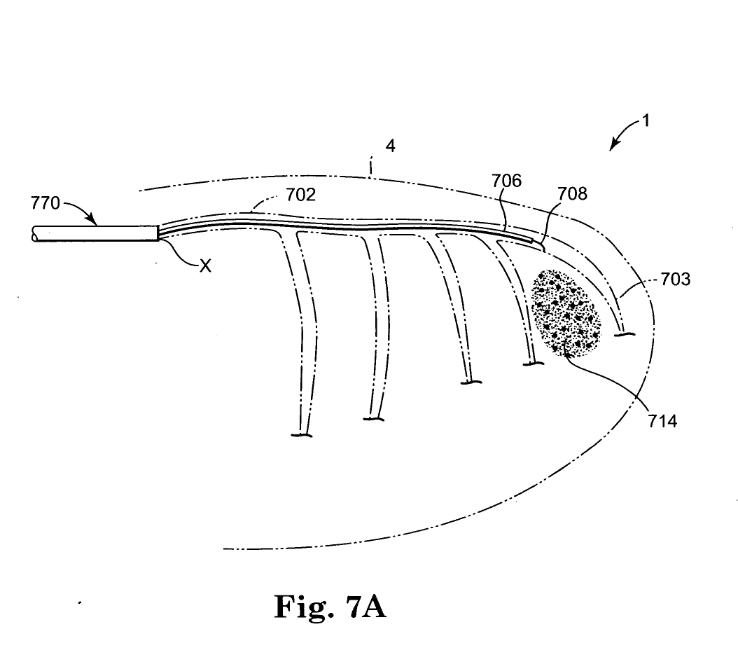

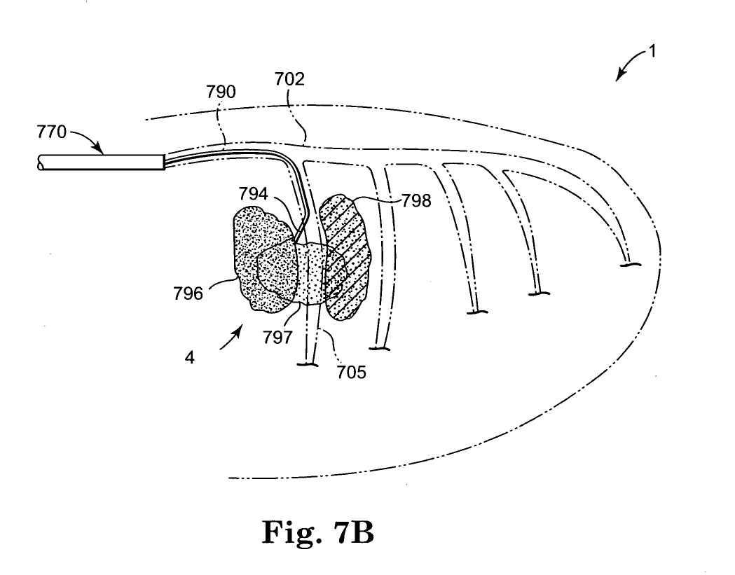

For further illustration, FIG. 7A shows a schematic view of a treatment wherein a delivery catheter 770 cannulates a coronary vessel 702 and delivers agent delivery device 706 to vessel 703 where needle 708 is advanced to penetrate and inject scaffolding material 714. As further illustrated by FIG. 7B, other vessels (e.g. vessel 705) may be cannulated in this manner, e.g. using guidewire tracking capabilities, and using mapping or other techniques different infarct regions may be located and treated, such as by forming sequential scaffolds 796, 797, 798 with agent delivery catheter 790 and injection needle 794. By repeat injections with a repositioned needle, or multiple injections with respective needles of an array assembly, such zones overlap to treat a wider area of damage. It is to be appreciated that the transvascular embodiments just described are illustrative and modifications may be made. For example, either balloon-assisted needles, or end-hole needle assemblies, or other equipment constructed for transvascular, extravascular scaffolding injection may be used according to the embodiments shown and discussed. Moreover, other uses of these particular devices, e.g. the balloon-based needle devices may be pursued, either according to similar designs as shown for the particular exemplary applications in the Figures, or with suitable modifications.

In further exemplary modifications, needles may be replaced by other modes for delivering the desired agent, such as through walls of porous membranes adapted to be engaged against tissue for delivery. Other devices than a balloon may be used as well, such as expandable members such as cages, or other devices such as loop-shaped elongate members that may be configured with appropriate dimension to form the desired area for delivery. Moreover, other regions than circular or partially circular (e.g. curvilinear) may be injected and still provide benefit without departing from the intended scope hereunder. In still further embodiments, those particular embodiments described above for injecting scaffolding within cardiac tissue may also be combined with various pacing devices, structures, and techniques. In one regard, the needle assemblies themselves may be used for pacing the region of the heart associated with the infarct or otherwise damaged zone treated with the injected scaffold. Or, devices may be used adjunctively as different assemblies though cooperating in overall cardiac healthcare. Further more detailed examples of devices & methods intended or otherwise adapted for pacing or other cardiac stimulation or electrical coupling are disclosed in the following documents: U.S. Pat. No. 4,399,818 issued Aug. 23, 1983 to Money; U.S. Pat. No. 5,683,447 issued Nov. 4, 1997 to Bush et al.; U.S. Pat. No. 5,728,140 issued Mar. 17, 1998 to Salo et al.; U.S. Pat. No. 6,101,410 issued Aug. 8, 2000 to Panescu et al.; U.S. Pat. No. 6,128,535 issued Oct. 3, 2000 to Maarse; US Patent Application Publication No. 2002/0035388 published Mar. 21, 2002 (Lindemans et al.); US Patent Application Publication No. 2002/0087089 published Jul. 4, 2002 in the name of Ben-Haim; WO 98/28039 published Jul. 2, 1998 in the name of Panescu et al.; WO 01/68814 published Oct. 20, 2001 in the name of Field; WO 02/22206 published Mar. 21, 2002 in the name of Lee; and WO 02/051495 published Jul. 4, 2002 in the name of Ideker et al., all of which are hereby incorporated by reference in their entirety.

Whereas FIGS. 7A and 7B show highly beneficial transvascular delivery of mixed scaffolding agent, respectively, into a ventricle wall, the delivery techniques may be combined for an overall result-in particular where different gauge needles or types of delivery devices are required for each component of a mixed scaffold. One precursor agent of a multiple-part scaffold may be accomplished for example transvascularly, in combination with a transcardiac approach with the other. Still further, whereas some agents may be delivered via a transcardiac delivery modality, other agents may also be delivered via the transvascular approach-each approach may provide for medical benefits at different areas of the ventricle wall, whereas their combination may provide a complete and still more beneficial medical result across the ventricle. To this end, the transcardiac approach is generally herein shown and described as the right heart system is often preferred for access. However, left ventricular transcardiac delivery of either or both of the polymer and cellular agents is also contemplated, instead of or in combination with the endo-ventricular approach (or transvascular approach). Any combination or sub-combination of these are contemplated.

Different volumes of scaffolding agent, and different numbers, sizes, patterns, and/or lengths of injection needles may be used to suit a particular need. In one regard, a prior diagnostic analysis may be used to determine the extent of the condition, location of the condition, or various anatomical considerations of the patient which parameters set forth the volume and/or pattern of scaffold agent or injection needle array to use for delivery. Or, a real time diagnostic approach may allow for stimulus or other effects to be monitored or mapped, such that the amount of agent, or distance, direction, or number of needle deployment, is modified until the correct result is achieved. Therefore, for example, the needles of such embodiments may be retractable and advanceable through tissue so that different arrangements may be tried until the damaged region is mapped and characterized for appropriate scaffolding injection.

It is further contemplated that the agent delivery and electrode embodiments, though highly beneficial in combination with each other, are independently beneficial and may be used to provide beneficial results without requiring the other.

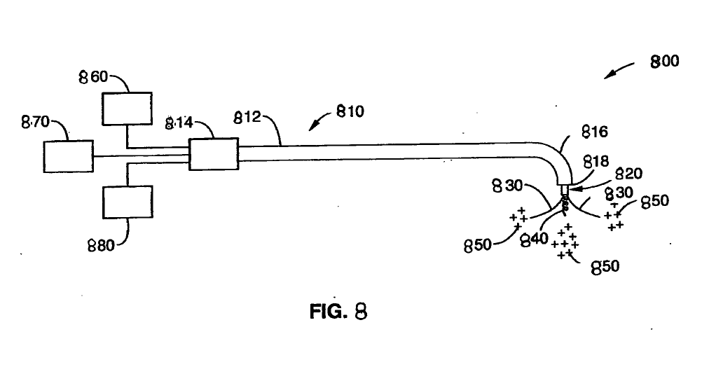

An example of a beneficial overall assembly is shown in FIG. 8. More specifically, intraventricular scaffolding system 800 is shown to include a delivery catheter 810 that cooperates to provide for both delivery of scaffolding materials 850 as well as electrode needles 830 and an anchor 840 as follows. Delivery catheter 810 has a proximal end portion 812 with a proximal coupler 814, distal end portion 816, and distal tip 818, and is an intracardiac delivery catheter adapted to deliver its contents toward the left ventricle wall from within the left ventricle chamber. Extendable from delivery catheter 810 is an inner catheter 820 with an extendable screw needle 840, and multiple spaced extendable electrode needles 830 spaced about screw needle 840. All or only some of central anchor 840, extendable electroded needles 830, and the tip of member 820 may be provided as stimulation electrodes to be coupled to energy source 860, such as via shaft 820. Moreover, all or only some of central screw 840, extendable electrode members 830, or tip of member 820, may be further adapted to deliver a volume of scaffolding agent into the region also coupled by the electrode sections, as shown at regions 850, such as via ports coupled to passageways (not shown) that are further coupled to a source of such scaffolding agent 870 (shown schematically).

This combination device is considered highly beneficial for stimulating substantial portions of the ventricle, such as for pacing and in particular treating left ventricular wall dysfunction. As further shown in FIG. 18 and illustrative of other embodiments providing extendable elements to be driven into tissue such as in the ventricle wall, a further device 880 may be coupled to such assembly that is an actuator that either allows for automated or manual extension of the respective extendable elements.

Further elements that may be provided in an overall system such as that shown in FIG. 8 or other embodiments herein, include monitoring sensors and related hardware and/or software, such as incorporated into or otherwise cooperating with an energy source such as a pacemaker/defibrillator, including for example: to map electrical heart signals for diagnostic use in determining the desired scaffolding result; and/or feedback control related to the effects of injecting the scaffolding itself, such as set points, etc.

Beads Having Other Therapeutic Properties

A variety of biological material may be delivered with injectable polymer-based beads 300, including cells such as stem cells, fibroblasts, or skeletal cells; proteins, plasmids, or genes; growth factors in either protein or plasmid form; chemo-attractants; fibrin fragment E; RDG binding sites; various pharmaceutical compositions; or other therapeutically beneficial materials; or any combination of the foregoing. The beneficial combination of RDG binding activity (or other cellular affinity factors) and fragment E (or other angiogenic factors), for example, may be achieved with beads.

Beads 300 may be made to encapsulate cells in the following manner. In one embodiment, calcium alginate polymers that can form ionic hydrogels may be sufficiently malleable to be used to encapsulate cells. The hydrogel is produced by cross-linking the anionic salt of alginic acid, a carbohydrate polymer isolated from seaweed, with calcium cations, whose strength increases with either increasing concentrations of calcium ions or alginate. The alginate solution may then be mixed with the cells to be implanted to form an alginate suspension. The suspension may then be injected directly into a patient prior to hardening of the suspension. The suspension may then harden over a short period of time due to the presence in vivo of physiological concentrations of calcium ions. Specific examples of formulations to form ionic hydrogels from calcium alginate polymers may be found in U.S. Pat. No. 6,281,015 issued Aug. 28, 2001 to Mooney et. al., which is included within this application as an appendix. In an alternative approach, peptide moieties (e.g., RGD or GREDVY) may be mixed in solution with the alginic acid allowing covalent bonding between the peptides and the alginates prior to mixing with the cells to be injected. In an alternative embodiment, alginate or chitosan beads may encapsulate cells which have previously been ionically entrapped by nanoparticles. One such arrangement of cells entrapped by nanoparticles can be found in published article by Mahoney and Saltzman entitled “Transplantation of Brain Cells Assembled Around a Programmable Synthetic Microenvironment” in Nature Biotechnology, Volume 19, 934-939, 2001, the disclosure of which is incorporated by reference in its entirety. The procedure for encapsulation may include the electrostatic bead generation method and apparatus mentioned earlier or the coaxial air driven microencapsulator apparatus as will be recognized by those skilled in the art upon review of the present disclosure. In another technique, alginate or chitosan beads may encapsulate cells dispersed in solution by way of a lypholizing (freeze drying) procedure utilizing a sufficient vacuum to crystallize the solution and entrap the cells. In this environment the freeze-dried beads may be temporarily packaged for shipment to a destination for their ultimate medical use wherein the beads may be re-hydrated prior to injection via hypodermic needle or air gun mist. In yet another technique, alginate beads may encapsulate cells by an emulsification/gelation process wherein an alginate solution containing an insoluble calcium salt is dispersed in oil, and gelation may be achieved by gentle acidification with an oil-soluble acid that causes calcium ion release. Specific examples of formulations to form alginate beads via the emulsification/gelation procedure may be found in published article “Microencapsulation of Hemoglobin in Chitosan-coated Alginate Microspheres Prepared by Emulsification/Internal Gelation,” AAPS Journal 2006, Vol 7. No. 4, Article 88, Jan. 13, 2006, by authors Caterina M. Silva et. al., the disclosure of which is incorporated by reference in its entirety. Microspheres with a mean diameter of less than 30 μm and an encapsulation efficiency of above 90 percent are attainable with this technique.

Other suitable materials having beneficial effects in such combination are also contemplated, such as other polymers or molecular scaffolds or materials that intervene sufficiently to inter-cellular gap junctions or otherwise impact the extracellular matrix in cardiac tissue structures to substantially enhance function and/or support of a damaged wall structure. Moreover, collagen or precursors or analogs or derivatives thereof are further considered useful for this purpose, either in addition or in the alternative to fibrin glue.

Beads 300 may contain or may be injected along with other materials, such as fluids or other substrates to provide the cells in an overall preparation as a cellular media that is adapted to be injected, such as in particular through a delivery lumen of a delivery catheter.

Beads 300 may contain or be injected with other synthetic polymers, such as polyethylene oxide (“PEO”), PEO-poly-1-lactic acid (“PLLA-PEO block copolymer”), poly (N-isopropylacrylamide-co-acrylic acid) (“poly (NIPAAm-co-Aac)”), pluronics, and poly-(N-vinyl-2-pyrrolidone) (“PVP”).

Beads 300 may be passivated with a coating such as sugar or a biopolymer, which is broken down when the beads are in situ in the heart by action of the body or by the use of an initiator combined and introduced with the passivated beads, or introduced into the same cardiac region as the passivated beads. Upon removal of the passivation coating, the surfaces of the beads are exposed so that the therapeutic effect of the beads may be realized.

Combining Beads with Other Scaffolding Materials

Among the various embodiments an injectable material is described that is adapted to form a therapeutic scaffolding in cardiac tissue structures. Beads may be embedded within the therapeutic scaffolding and released as the scaffolding is adsorbed. Examples of highly beneficial materials for use according to the invention include: cells, polymers, or other fluids or preparations that provide interstitial or other forms of internal wall support, such as stiffening inter-cellular junction areas. Fibrin glue agent has been identified as a highly beneficial biopolymer for such use. Another example includes an injectable material containing collagen, or a precursor or analog or derivative thereof.

Therapeutically effective scaffolding may be made from fibrin glue. Fibrin glue is an FDA approved biomaterial that is routinely used as a surgical adhesive and sealant. This biopolymer is formed by the addition of thrombin to fibrinogen. Thrombin in a kit is an initiator or catalyst which enzymatically cleaves fibrinogen which alters the charge and conformation of the molecule, forming a fibrin monomer. The fibrin monomers then proceed to aggregate forming the biopolymer fibrin. After combination of the two thrombin and fibrinogen components, the solution remains liquid for several seconds before polymerizing. Fibrin glue agent, either immediately following mixture of the precursor materials, or by delivering the materials separately to mix in-situ, is therefore adapted to be delivered to the myocardium via injection catheters or other injectors, thus requiring only a minimally invasive procedure. It is also biocompatible and non-toxic, without inducing inflammation, foreign body reactions, tissue necrosis or extensive fibrosis. [099

As a support, fibrin glue may be modified to tailor its mechanical properties for this particular application. An increase in thrombin or fibrinogen concentration results in an increase in tensile strength and Young's modulus. An increase in fibrinogen concentration will also decrease the degradation rate of the biopolymer.

Fibrin glue according is believed to act as an internal wall support (i.e. within the wall) to preserve cardiac function. During the initial stage in MI, matrix metalloproteases are upregulated which results in degradation of the extracellular matrix (ECM). This ECM degradation leads to weakening of the infarct wall and slippage of the myocytes leading to LV aneurysm. In addition, negative ventricular remodeling has been observed to typically continue until the tensile strength of the collagen scar strengthens the infarct wall.

Fibrin glue administration during the initial stage of an infarct is believed to increase the mechanical strength of the infarct region before the collagen scar has had to time to fully develop. Furthermore, fibrin glue adheres to various substrates including collagen and cell surface receptors (predominately integrins) through covalent bonds, hydrogen and other electrostatic bonds, and mechanical interlocking. Therefore, it is further believed that the fibrin glue prevents myocyte slippage and subsequent aneurysm by binding to the neighboring normal myocardium. Still further, it is also believed that injection of fibrin glue results in an upregulation or release of certain growth factors such as angiogenic growth factors which are known to improve cardiac function.

The fibrin scaffold provides an internal support to prevent LV expansion and prevents a decline in cardiac function. Fibrin glue solidifies inside the myocardium and provides an internal wall support believed preferable to external patches which have been used to prevent LV dilation. Furthermore, fibrin glue adheres to various substrates including collagen and cell surface receptors through covalent bonds, hydrogen and other electrostatic bonds, and mechanical interlocking. Therefore, it may prevent myocyte slippage and subsequent LV expansion by binding to the neighboring normal myocardium. Fibrin may also preserve LV function by increasing blood flow to the ischemic tissue. Similar to when delivered in an acute MI, fibrin glue also increased neovasculature formation compared to injection of BSA in our chronic MI model. Natively, fibrin is highly involved in wound healing and acts as the body's natural matrix for neovasculature formation.

Fibrin glue is observed to be generally biocompatible, non-toxic, and not generally observed to induce inflammation, foreign body reactions, tissue necrosis or extensive fibrosis. Another benefit of this injectable scaffold is that it is an already FDA approved material, which is routinely used as a surgical adhesive and sealant. Since it remains liquid before combination of its two components, it could also be delivered via catheter, thus requiring only a minimally invasive procedure in humans.

Benefits of Beads Embedded Within a Fibrin Glue Scaffold

Beads may be included in either the thrombin or fibrogen components of fibrin glue, or in both components. Depending on the type of beads, therapeutically beneficial results in addition to those provided by the fibrin glue scaffold alone may be realized. The beads may encapsulate cells such as skeletal myoblasts, which protects the myoblasts and improves cell survival during injection. The combination of skeletal myoblasts and fibrin glue significantly increased cardiac function and significantly decreased LV expansion compared to BSA, fibrin glue alone, and myoblasts in BSA. In addition to the favorable effects of fibrin alone, myoblasts in fibrin glue may have added benefit by increasing the myoblast density in the infarct area, particularly as the fibrin glue scaffold breaks down.

While injection of myoblasts with fibrin glue enhances cell transplant survival, there is a possibility that cell retention in infarcted myocardium may not be enhanced. However, encapsulating the myoblasts in beads may aid in retention, either due to the mechanical size of the beads or to the bonding properties imparted to the beads. In this way, not only is cell survival enhanced, but the initial population of cells at the site of injection may be increased, thereby increasing the therapeutically beneficial effect of the introduced cells.

Some applications may benefit from prolonging the presence of the scaffold. Where the scaffold is fibrin, for example, the fibrin is resorbed by enzymatic and phagocytic pathways so that a fibrin scaffold may disappear on the order of four weeks post-injection, or so. The short duration may not be sufficient where positive remodeling is desired, as where the infarct is extensive and significant negative remodeling has already occurred. In such applications, a simple fibrin glue matrix created by injection of the two components into the infarct may biodegrade before the desired therapeutic effect is attained.

One approach is to encapsulate the two components of fibrin glue, or of a scaffolding agent having a biopolymer capable of cross-linking such as an alginate or alginate-containing material and a cross-linking initiator, and inject the beads with the fibrin glue. As the in situ scaffold biodegrades, the exposed beads also biodegrade, thereby releasing their material which in turn forms new scaffolding. Alternatively, a mixture of instantly biodegradable beads and more slowly biodegradable beads may be injected, so that the instantly biodegradable beads immediately release their material to form an initial scaffold that is maintained over time by materials from the more slowly deteriorating beads.

Materials Described Herein Generally Illustrate Broader Classes of Materials

The materials described herein generally illustrate certain broader classes of materials, which classes may contribute additional alternatives as would be apparent to one of ordinary skill. Where a compound is herein identified in relation to one or more embodiments described herein, such as for example collagen or fibrin, precursors or analogs or derivatives thereof are further contemplated. For example, material structures that are metabolized or otherwise altered within the body to form such compound are contemplated. Or, combination materials that react to form such compound are also contemplated. Additional materials that are also contemplated are those which have molecular structures that vary insubstantial to that of such designated compounds, or otherwise have bioactivity substantially similar thereto with respect to the intended uses contemplated herein (e.g. removing or altering non-functional groups with respect to such bioactive function). Such group of compounds, and such precursors or analogs or derivatives thereof, is herein referred to as a “compound agent.” Similarly, reference herein to other forms of “agents”, such as for example “polymer agent” or “fibrin glue agent” may further include the actual final product, e.g. polymer or fibrin glue, respectively, or one or more respective precursor materials delivered together or in a coordinated manner to form the resulting material.

It is to be appreciated that where fibrin glue or related agents are herein described, it is further contemplated that other materials such as collagen, or precursors or analogs or derivatives thereof, may also be used in such circumstances, in particular relation to forming injected scaffolding, either alone or in combination with cells.

The term “protein” is intended to include a wide variety of proteins. Another example of a suitable protein is integrin, which has been observed to enhance cellular binding and thus may be injected into cardiac tissue structures to provide substantial benefit to cellular tissue formation and/or retention there. For further illustration, further particular embodiments may also include integrin in combination with cell delivery, and/or in combination with others of the non-living compounds herein described.

Injectable Biopolymer-Based Beads Suitable for Conduction Modification

Cell types which produce gap junctions in recipient hearts, including fetal cardiomyocytes, adult bone marrow stem cells, or fibroblasts or myoblasts or other cell types modified to express sufficient connexins, such as Connexin-43, are may be delivered to the myocardium in a suitable biopolymer bead, with the aims of improving both contractility and preventing remodeling. More specific modes of the invention using cells include myoblasts, fibroblasts, stem cells, or other suitable cells that provide sufficient gap junction conduction with cardiac cells to form the desired conductive coupling to the surrounding cardiac structure to provide for improved chamber conduction and contraction. In other modes, where such coupling is not achieved sufficient to provide for proper sinus rhythm through the injected region, the opposite may be desired. In other words, complete decoupling of the injected region may be preferred in order to reduce a potential “pro-arrhythmic” risk of existing, yet incomplete, contractile conduction through or from the injected zone. With further respect to cell delivery, they may be cultured from the patient's own cells, or may be exogenous and foreign to the body, such as from a regulated cell culture.

Use of myoblast transplantation according to certain aspects and modes of the present invention adapts delivery of these cells in a highly localized manner at locations along infarct regions otherwise often uncoupled to the cardiac cycle, thus gap junction results between the injected and resident cells may not be substantially relevant to intended medical results.

Fibroblasts are another alternative cell of the type considered highly beneficial for delivery with beads. The electrophysiological properties of fibroblasts are fairly consistent from one fibroblast to the next, and are believed to be effective for consistent effects on conduction. Therefore, in one illustrative embodiment using fibroblasts delivered to ventricular wall dysfunction or ischemia, very similar responses can be predicted between batches/injections. Therefore damaged myocardium may be treated using fibroblast cell transplantation with beads. According to a highly beneficial variation of such embodiment, such fibroblasts are autologous, typically taken from dermal samples, and are subsequently prepared appropriately and transplanted to a location within a cardiac tissue structure to facilitate treatment of cardiac injury, such as infarct, ischemia, and/or cardiomyopathy and CHF.

Other materials and methods may also be employed to include the production of gap junction proteins in fibroblast cells in order to normalize the conduction pathway via the ability of the fibroblasts to electromechanically couple with the existing cardiac myocytes surrounding the injected scaffold zone.

Injectable Hydrogel Agents

Injectable materials may be used to form alginate and chitosan hydrogels to supply mechanical integrity for interstitial scaffolding, to retain various other materials in place, for conduction modification, and so forth. Alginate hydrogels may be formed using either or both G-rich and M-rich alginate materials in the presence of divalent cations such Ca²⁺, Ba²⁺, Mg²⁺, or Sr²⁺. Gelling occurs when the divalent cations take part in ionic binding between blocks in the polymer chain, giving rise to a 3 dimensional network. In one approach, a dual chamber syringe converging into a single lumen injection needle may be used to inject the mixed components of the alginate mixture to gel in-vivo. One component may be a sodium alginate fully solublized in an aqueous solution such as H₂0. The other component may be one of the divalent cations mentioned above dispersed (not dissolved) in solution. The compounds may be mixed in any suitable manner. Prior to injection, for example, a T-type adapter attached to the syringe may be set to provide mixing of the components and initiate the gelling action, and then set to allow the alginate mixture undergoing gelling to enter the lumen and to be injected into the cardiac tissue of interest. The alginate mixture may be injected immediately, or may be allowed to partially pre-cure in the syringe in order to increase the viscosity of the hydrogel prior to injection. In some instances, a pre-cured formulation may reduce the possibility that a less viscous hydrogel may diffuse or migrate away from the tissue area of interest after injection. In order to limit or minimize diffusion/migration away from the injection site, it would be beneficial to utilize alginate materials with molecular weights in excess of about 300,000. In another approach, the sodium alginate solution and dispersed cation may be pre-mixed in an external mixing chamber, and aspirated into a single lumen syringe from which it may be injected into the cardiac tissue of interest. In another approach, the sodium alginate solution may be pre-mixed with an appropriate peptide (e.g., RGD or GREDVY) for covalent attachment of the peptide to the alginate prior to mixing with the divalent cations. In addition to providing mechanical integrity for interstitial scaffolding, alginate hydrogels with covalently attached peptides may enhance cell proliferation in MI damaged cardiac tissue.

Experiment 1: Testing the Effects of GRGDSP on Human Umbilical Endothelial Vein Cells (HUVEC) on Proliferation.

In one in-vitro experiment, human umbilical vein endothelial cells (HUVEC) were utilized over a 10 day gestation period to demonstrate this effect. In this study, GRGDSP peptide material was covalently attached to high molecular weight M-type alginate (MW 297,000) in a ratio of 12 peptides per alginate molecule. HUVEC cells were added to the alginate solution and the solution was caused to gel by addition of 102 millimolar CaCl₂. HUVEC cells were also added to a negative control high molecular weight alginate solution without peptide attachment and caused to gel via addition of calcium chloride as before. Both gels were measured for density at day one via an optical absorption measurement at 490 nanometers and again at day 10. The negative control alginate w/o peptide showed a marginal increase in absorption from 0.4 to approximately 0.42 absorption units at day 10 indicating a small increase in cell population, whereas the peptide attached alginate increased from 0.4 to 1.0 absorption units (a 2.5× increase) over the same time period. Given that optical absorption units (Absorbance) are logarithmic in nature a 2.5× enhancement is significant (10^{2.5}≈316). For optimum cell proliferation in human endothelial I tissue, the peptide to alginate ratio may require clinical investigation, however the above results demonstrate promising in-vitro feasibility.

Experiment 2: Testing the Effects of RGD on Human Umbilical Endothelial Vein Cells (HUVEC) on Proliferation.

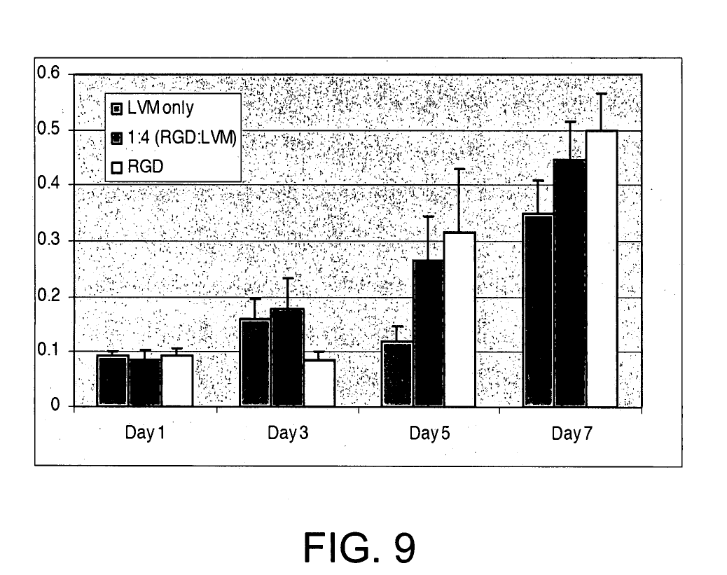

Pooled human umbilical vein endothelial cells (HUVECs) cultured in EBM-2 (supplemented with Singlequots and 5% FBS) and used no later than passage 3. Cells were plated on solid culture medium and grown for seven days. Once set of plates included Low Viscosity Mannuronic Acid (LVM), a second set of plates included the RGD peptide and LVM at a 1:4 ratio, and the third set of plates included only the RGD peptide. Cell counts were taken on alternating days. The results are graphically shown in FIG. 9. The graph illustrates the tendency of RGD to promote HUVEC proliferation in culture.

Experiment 3: Effects of RGD-Alginate on Human Mesenchymal Stem Cell (MSC) Adhesion

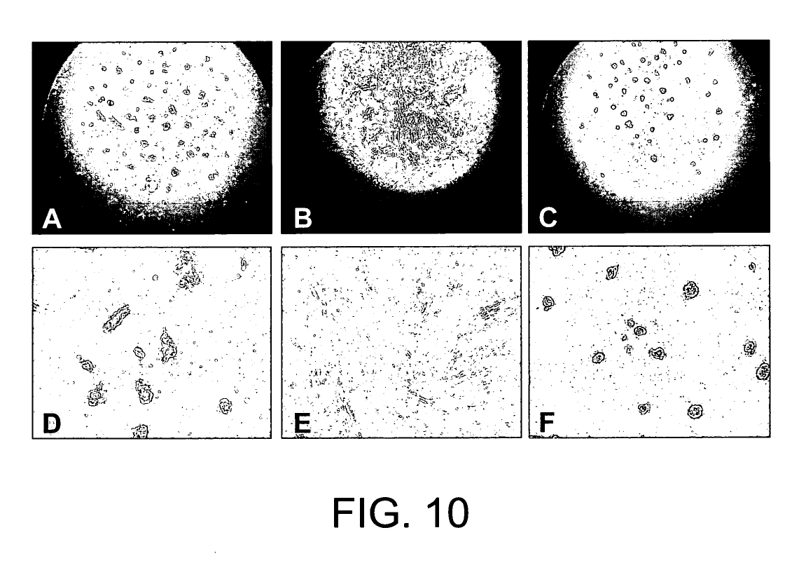

Bone marrow-derived human mesenchymal stem cells (Cambrex, Walkersville, Md.) were cultured in Mesenchymal Stem Cell Growth Medium (MSCGM, Cambrex) with 1% penicillin/streptomycin. Cells were subcultured every 5-7 days and used within 8 passages. For in vitro characterization, 1.5×10⁵ cells were grown on either non-modified alginate, RGD modified alginate or VAPG modified alginate coated tissue culture dishes in MSCGM. 1.5% alginate solution was made from dissolving a high mannuronic acid (M units) alginate (ProNova LVM, FMC Biopolymer, Norway) in 0.9% NaCl. Alginate gel formation was based on the addition of the cross-linker solution, 102 mM CaCl₂ FIG. 10 illustrates the ability of MSC to adhere to RGD-alginate (panel E) but not to alginate (panel d) or VAPG-alginate (panel F). Each photograph illustrates the In Vitro culture of MSCs after 48 hrs. Plate A shows the MSCs grown on non-modified alginate. Plate B shows the MSCs grown on RGD modified alginate. Plate C shows the MSCs grown on VAPG modified alginate. Plate D shows the MSCs of Plate A grown on non-modified alginate at a higher magnification. Plate E shows the MSCs of Plate B grown on RGD modified alginate at a higher magnification. Plate F shows MSCs of Plate C the grown on VAPG modified alginate at a higher magnification. The results of this study demonstrates that RGD-alginate promotes cell adhesion while MSC do not adhere to either alginate or VAPG-alginate coated plates.

Experiment 4: Induction of growth factors by RGD

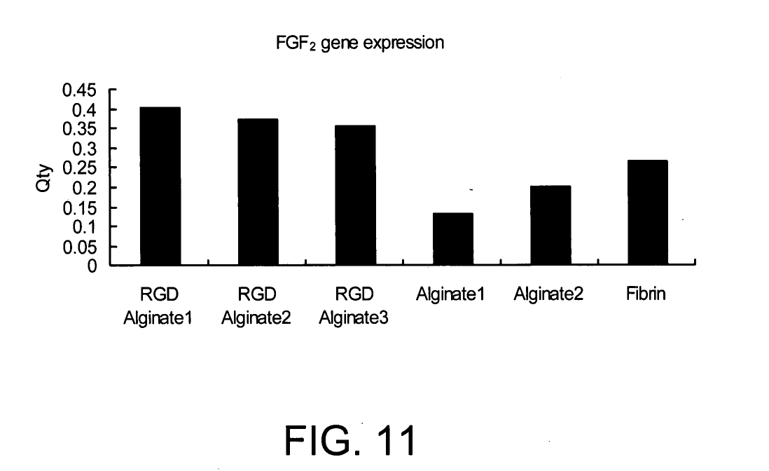

Cells were cultured for 5 days on either fibrin-coated, alginate or RGD-alginate substrates (control) before lysing with Trizol (Invitrogen, Carlsbad, Calif.). RNA isolation and qPCR was carried out according to previous literature. Primers for qPCR were designed by ABI Prism Primer Express software (Applied Biosystems), (forward primer: CCAGTAATCTTCCATCTTCCTTCATAG; reverse primer: CACATCAAGCTACAACTTCAAGCA). The mRNA expression was normalized by 18S. The data is presented as fold change, the ratio of normalized mRNA quantities [(MSCs on fibrin substrate)/(MSCs on non-coated substrate)]. FIG. 11 is a bar graph illustrating the quantity of mRNA expressed by each group. This study of three separate RGD-alginate samples demonstrates enhanced production of the angiogenesis growth factor, FGF2 gene expression.

Experiment 5: In Vivo Study was Carried Out to Investigate Whether the Modified Alginate Can Repair a Chronic Myocardial Aneurysm and Stimulate Angiogenesis

To test the hypothesis that alginate or RGD-alginate scaffold expands the thinned wall of the anuerysmal left ventrical, restores left ventricular geometry and induces angiogenesis, Sprague-Dawley rats underwent left coronary artery (LAD) occlusion for 20 minutes, followed by reperfusion. Five weeks following infarction, at which time the remodeling process is largely complete, injections of either the control 0.5% bovine serum albumin (BSA) in phosphate buffered saline (PBS) (n=5, alginate (n=6) or RGD-alginate (n=6) were made directly into the infarcted myocardium. All injections were made through 27-guage needles into the infarcted area of the left ventrical. The infarcted area was identified by a darker region of left ventricular wall with reduced contractility, mostly within anterior wall. The control and experimental groups were sacrificed 24 hours after injection in order to examine the location and structural effect of the polymer injections compared to control. 1.5% alginate solution was made from dissolving a high mannuronic acid (M units) alginate (ProNova LVM, FMC Biopolymer, Norway) in 0.9% NaCl. Alginate gel formation was based on the addition of the cross-linker solution, 102 mM CaCl₂ [16]. Transthoracic echocardiography was performed on all animals under anesthesia of isoflurane (2 L/min) five weeks after MI as a baseline echocardiogram. Follow-up echocardiograms were performed 2 days and 5 weeks after injection (10 weeks after MI).

The echocardiography results showed that both modified and non-modified alginate significantly restored left ventricle geometry, increased left ventricular wall thickness, and significantly improved cardiac function 5 weeks post injection of biopolymers. Immunofluorescence staining showed that both alginates enhanced angiogenesis compared to saline injected group. The modified alginate had higher arteriole density in infarcted area than non-modified group, indicating that cell recognition ligands affect the microenvironment of ischemic myocardium and increases arteriogenesis.

Experiment 6: Creation of Microspheres

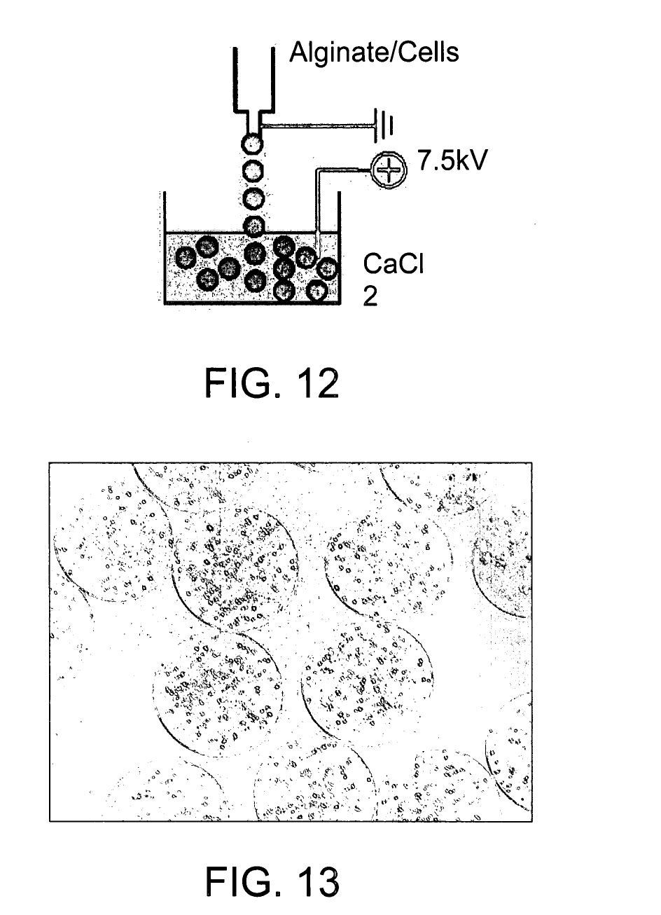

Creation of microspheres was performed by passing 2% LVM alginate or RGD-alginate through a nozzle tip in an electrostatic field. Utilizing a modified 30 gauge needle, microsphere of approximately 75-100 μm diameter were made as illustrated in FIG. 12. Microspheres were made alone and by adding either MSCs or fibroblasts with the alginate, encapsulation of either MSCs or fibroblasts was achieved. The protocol for encapsulating the MSCs consisted of 2% w/v Alginate solution was made by dissolving alginate LVM and RGD-peptide modified alginate (LVM:RGD modified alginate=5:1, both from NovaMatrix) in Mesenchymal Stem Cell Growth Medium (Cambrex) using a sonicator (VWR, model 75T) for 2 hr and stored at 4 degrees Celsius before use. MSC (Cambrex) cell suspension was added to alginate solution to yield a final cell density of 3×10⁶/ml. The MSC alginate solution in syringe pump was connected electrostatic bead generator (Nisco, Switzerland). Alginate beads were generated with flow rate 10 ml/hr, voltage 7.5 kV, nozzle 30-33 gauge, gelling bath solution CaCl₂ concentration 102 mM, resulting in beads size 75-100 μm in diameter. After beads formation, CaCl₂ solution was removed and beads were washed with HEPES. Beads were then surface coated with poly-L-Lysine solution for 2 min and washed with HEPES for 2 times. After washing, HEPES solution was then replaced with MSCGM and beads suspension was cultured in tissue culture flask for future study. The beads including MSCs are shown in FIG. 13.

To determine the cell proliferation and viability, beads were depolymerized by soaking beads in depolymerization solution containing 100 mM sodium citrate (Fisher Scientific), 10 mM MOPS(Sigma) and 27 mM NaCl for 30 minutes at 37 degrees. The solution was centrifuged at 1200 rpm for 10 min. The cell pellet was resuspended in medium and cell density/viability was determined by trypan blue staining. Viability of the fibroblasts was demonstrated by staining with Trypan blue stain. It was determined that cell viability was greater than 99% at 2 weeks.

To determine whether the microspheres are suitable candidates for application into the myocardium via a catheter, the microspheres were injected through 27 gauge, 25 gauge and 21 gauge needles. It was found that microsphere shearing occurred in 20% of microspheres injected through a 27 gauge needle, while there was no destruction of microsphere injected through a 25 or 21 gauge needle. Microspheres were then injected through a long injection catheter with a 27 gauge needle to test whether the microspheres could be applied via a long vascular injection catheter. It was found that >80% of the microspheres were intact, thus this size of microsphere would be suitable for potential delivery of microspheres to injured human myocardium.

Experiment 7: In Vivo Testing of Microspheres

To test whether alginate and/or RGD-alginate microspheres have the ability to reshape an infarcted myocardium and improve left ventricular function, Sprague-Dawley rats underwent left coronary artery (LAD) occlusion for 20 minutes, followed by reperfusion. Five weeks following infarction, at which time the remodeling process is largely complete, injections of either the control 0.5% bovine serum albumin (BSA) in phosphate buffered saline (PBS) (n=6, alginate microspheres (n=7) or RGD-alginate microspheres (n=7) were made directly into the infarcted myocardium. All injections were made through 27-guage needles into the infarcted area of the left ventrical. The infarcted area was identified by a darker region of left ventricular wall with reduced contractility, mostly within anterior wall. Alginate and RGD-alginate microspheres were made as described in experiment 5. Transthoracic echocardiography was performed on all animals under anesthesia of isoflurane (2 L/min) five weeks after myocardial infarction as a baseline echocardiogram. Follow-up echocardiograms were performed 2 days after injection.

The echocardiography results showed that both modified and non-modified alginate significantly restored left ventricle geometry, increased left ventricular wall thickness, and significantly improved cardiac function 2 days post injection of biopolymers.

Although this written description contains many details, these details should not be construed as limiting the scope of the invention as set forth in the following claims, but should instead been seen as merely providing illustrations of various embodiments of this invention. Therefore, it will be appreciated that the scope of the present invention fully encompasses many variations and modifications of the embodiments described herein. Embodiments that include a description of a single element are not to be limited to one and only one such element. All structural, chemical, and functional equivalents to the elements of the described embodiments are to be considered within the scope of the invention. Moreover, it is not necessary for an apparatus or method to address each and every problem sought to be solved by the present invention, for it to be encompassed by the present invention. It will be appreciated that the apparatus may vary as to configuration and as to details of the parts, and that the method may vary as to the specific steps and sequence, without departing from the present invention.

1. A kit for treating a heart in a diseased condition comprising:
a source of a bead-containing agent; and
an agent delivery system for delivering a therapeutically effective amount of the bead-containing agent from the source to a myocardial region of the heart that relates to the diseased condition, the agent delivery system comprising:
a proximal portion for coupling to the source; and
a distal portion for introducing the bead-containing into or in proximity to the myocardial region;
wherein the bead-containing agent comprises a plurality of beads; and
wherein each of the beads comprises a core comprising a plurality of alginate polymers.

2-11. (canceled)

12. A kit for treating a heart in a diseased condition comprising:
a source of a bead-containing agent; and
an agent delivery system for delivering a therapeutically effective amount of the bead-containing agent from the source to a myocardial region of the heart that relates to the diseased condition, the agent delivery system comprising:
a proximal portion for coupling to the source; and
a distal portion for introducing the bead-containing into or in proximity to the myocardial region;
wherein the bead-containing agent comprises a plurality of beads; and
wherein each of the beads comprises a core comprising a plurality of chitosan polymers.

13-14. (canceled)

15. A kit for treating cardiac infarction in a heart, comprising:
a source of a bead-containing agent; and
an agent delivery system for delivering a therapeutically effective amount of the bead-containing agent from the source to interstitial spaces of a infarcted myocardial region of the heart, the agent delivery system comprising:
a proximal portion for coupling to the source; and
a distal portion for introducing the bead-containing into or in proximity to the infarcted myocardial region;
wherein the bead-containing agent comprises a plurality of beads having a myocardium-adherent property for lodging within the interstitial spaces to provide structural support to the infarcted myocardial region.

16-19. (canceled)

20. A kit for treating cardiac arrhythmia in a heart, comprising:
a source of a bead-containing agent; and
an agent delivery system for delivering a therapeutically effective amount of the bead-containing agent from the source to a myocardial region of the heart having electrical activity relating to the cardiac arrhythmia, the agent delivery system comprising:
a proximal portion for coupling to the source; and
a distal portion for introducing the bead-containing into or in proximity to the myocardial region;
wherein the bead-containing agent comprises a plurality of beads having a conduction-modifying property for modifying the electrical activity in the myocardial region.

21-24. (canceled)

25. A kit for treating a heart in a diseased condition, comprising:
a source of a bead-containing agent; and
an agent delivery system for delivering a therapeutically effective amount of the bead-containing agent from the source to a myocardial region of the heart relating to the diseased condition, the agent delivery system comprising:
a proximal portion for coupling to the source; and
a distal portion for introducing the bead-containing into or in proximity to the myocardial region;
wherein the bead-containing agent comprises a plurality of beads, each encapsulating biological material selected from the group consisting of a cell, a gene, a peptide, a polypeptide, a protein, a neo-tissue, and any combination of one or more of the foregoing.

26-29. (canceled)

30. A kit for treating a heart in a diseased condition, comprising:
a source of a bead-containing agent; and
an agent delivery system for delivering a therapeutically effective amount of the bead-containing agent from the source to a myocardial region of the heart relating to the diseased condition, the agent delivery system comprising:
a proximal portion for coupling to the source; and
a distal portion for introducing the bead-containing into or in proximity to the myocardial region;
wherein the bead-containing agent comprises a plurality of beads, each encapsulating biological material selected from the group consisting of a cell, a gene, a peptide, a polypeptide, a protein, a neo-tissue, and any combination of one or more of the foregoing; and
wherein each of the beads has a myocardium-adherent property for lodging within interstitial spaces of the myocardial region to provide structural support thereto.

31-35. (canceled)

36. A kit for treating a heart in a diseased condition, comprising:
a source of a multiple-component agent; and
an agent delivery system for delivering a therapeutically effective amount of the multiple-component agent from the source to a myocardial region of the heart relating to the diseased condition, the agent delivery system comprising:
a proximal portion for coupling to the source; and
a distal portion for introducing the multiple-component agent into or in proximity to the myocardial region;
wherein the multiple-component agent comprises:
a first component;
a second component for contributing to the therapeutic effect in conjunction with the first component; and
a plurality of beads dispersed in at least one of the first and second components.

37-46. (canceled)

47. A kit for treating a heart in a diseased condition, comprising:
a source of a bead-containing agent; and
an agent delivery system for delivering a therapeutically effective amount of the bead-containing agent from the source to a myocardial region of the heart relating to the diseased condition, the agent delivery system comprising:
a proximal portion for coupling to the source; and
a distal portion for introducing the bead-containing into or in proximity to the myocardial region;
wherein the bead-containing agent comprises a cell-recruiting material.

48-49. (canceled)

50. A kit for treating a heart in a diseased condition, comprising:
a source of a bead-containing agent; and
an agent delivery system for delivering a therapeutically effective amount of the bead-containing agent from the source to a myocardial region of the heart relating to the diseased condition, the agent delivery system comprising:
a proximal portion for coupling to the source; and
a distal portion for introducing the bead-containing into or in proximity to the myocardial region;
wherein the bead-containing agent comprises an angiogenic-initiating material.

51-52. (canceled)

53. A kit for treating a heart in a diseased condition, comprising:
a source of a bead-containing agent; and
an agent delivery system for delivering a therapeutically effective amount of the bead-containing agent from the source to a myocardial region of the heart relating to the diseased condition, the agent delivery system comprising:
a proximal portion for coupling to the source; and
a distal portion for introducing the bead-containing agent into or in proximity to the myocardial region;
wherein the bead-containing agent comprises one or more materials having cell-recruiting and angiogenic-initiating properties.

54-55. (canceled)

56. A kit for treating a heart in a diseased condition, comprising:
a source of a multiple-component agent; and
an agent delivery system for delivering a therapeutically effective amount of the multiple-component agent from the source to a myocardial region of the heart relating to the diseased condition, the agent delivery system comprising:
a proximal portion for coupling to the source; and
a distal portion for introducing the multiple-component agent into or in proximity to the myocardial region;
wherein the multiple-component agent comprises:
a first component comprising a sodium alginate fully solublized in an aqueous solution; and
a second component comprising divalent cations dispersed in solution;
and wherein the first component and the second component interact to contribute to a therapeutic effect.

57-60. (canceled)

61. A kit for treating a heart in a diseased condition, comprising:
a source of a hydrogel agent; and
an agent delivery system for delivering a therapeutically effective amount of the hydrogel agent from the source to a myocardial region of the heart relating to the diseased condition, the agent delivery system comprising:
a proximal portion for coupling to the source; and
a distal portion for introducing the hydrogel agent into or in proximity to the myocardial region;
wherein the hydrogel agent comprises alginate polymers and peptides adapted for covalent bonding to the alginate polymers.

62-64. (canceled)

65. A method for treating a heart condition, comprising:
identifying a myocardial region of the heart relating to the heart condition; and applying a therapeutically effective amount of a bead-containing agent at least in proximity to the myocardial region;
wherein the bead-containing agent comprises a plurality of beads; and wherein each of the beads comprises a core comprising a plurality of alginate polymers.

66-78. (canceled)

79. A method for treating a heart condition, comprising:
identifying a myocardial region of the heart relating to the heart condition; and
applying a therapeutically effective amount of a bead-containing agent at least in proximity to the myocardial region;
wherein the bead-containing agent comprises a plurality of beads; and
wherein each of the beads comprises a core comprising a plurality of chitosan polymers.

80-84. (canceled)

85. A method for treating a cardiac infarction, comprising:
identifying an infarcted myocardial region of the heart; and
applying a therapeutically effective amount of a bead-containing agent into interstitial spaces of the infarcted myocardial region;
wherein the bead-containing agent comprises a plurality of beads having a myocardium-adherent property for lodging within the interstitial spaces to provide structural support to the infarcted myocardial region.

86-88. (canceled)

89. A method for treating a cardiac arrhythmia, comprising:
identifying a myocardial region of the heart relating to electrical activity of the heart; and
applying a therapeutically effective amount of a bead-containing agent into the identified myocardial region;
wherein the bead-containing agent comprises a plurality of beads having a conduction-modifying property for modifying the electrical activity of the heart in the identified myocardial region.

90-92. (canceled)

93. A method for treating a heart condition, comprising:
identifying a myocardial region of the heart relating to the heart condition; and
applying a therapeutically effective amount of a bead-containing agent at least in proximity to the myocardial region;
wherein the bead-containing agent comprises a plurality of beads, each encapsulating biological material selected from the group consisting of a cell, a gene, a peptide, a polypeptide, a protein, a neo-tissue, and any combination of one or more of the foregoing.

94-96. (canceled)

97. A method for treating a heart condition, comprising:
identifying a myocardial region of the heart relating to the heart condition; and
applying a therapeutically effective amount of a bead-containing agent at least in proximity to the myocardial region, the bead-containing agent comprising a plurality of beads;
wherein each of the beads has a myocardium-adherent property for lodging within the interstitial spaces to provide structural support to the infarcted myocardial region; and
wherein each of the beads encapsulates biological material selected from the group consisting of a cell, a gene, a peptide, a polypeptide, a protein, a neo-tissue, and any combination of one or more of the foregoing.

98-101. (canceled)

102. A method for treating a heart condition, comprising:
identifying a myocardial region of the heart relating to the heart condition; and
applying a therapeutically effective amount of a multiple-component agent at least in proximity to the myocardial region;
wherein the multiple-component agent comprises:
a first component;
a second component for contributing to the therapeutic effect in conjunction with the first component; and
a plurality of beads dispersed in at least one of the first and second components.

103-111. (canceled)

112. A method for treating a heart condition, comprising:
identifying a myocardial region of the heart relating to the heart condition; and
applying a therapeutically effective amount of a bead-containing agent at least in proximity to the myocardial region;
wherein the bead-containing agent comprises a cell-recruiting material.

113. (canceled)

114. A method for treating a heart condition, comprising:
identifying a myocardial region of the heart relating to the heart condition; and
applying a therapeutically effective amount of a bead-containing agent at least in proximity to the myocardial region;
wherein the bead-containing agent comprises an angiogenic-initiating material.

115. (canceled)

116. A method for treating a heart condition, comprising:
identifying a myocardial region of the heart relating to the heart condition; and
applying a therapeutically effective amount of a bead-containing agent at least in proximity to the myocardial region;
wherein the bead-containing agent comprises one or more materials having cell-recruiting and angiogenic-initiating properties.

117. (canceled)

118. A method for treating a heart condition, comprising:
identifying a myocardial region of the heart relating to the heart condition; and
applying a therapeutically effective amount of a multiple-component agent at least in proximity to the myocardial region;
wherein the multiple-component agent comprises:
a first component comprising a sodium alginate fully solublized in an aqueous solution; and
a second component comprising divalent cations dispersed in solution;
and wherein the first component and the second component interact to contribute to a therapeutic effect.

119-121. (canceled)

122. A system for treating a heart condition, comprising:
a source comprising a bead-containing agent; and
an applicator for applying a therapeutically effective amount of the bead-containing agent from the container at least in proximity to an identified myocardial region of the heart relating to the heart condition;
wherein the bead-containing agent comprises a plurality of beads, each having a mean diameter of from about 30 μm to about 500 μm and comprising a core comprising a plurality of alginate polymers.

123-127. (canceled)

128. A method for treating a heart condition, comprising:
identifying a myocardial region of the heart relating to the heart condition; and
applying a therapeutically effective amount of a hydrogel agent at least in proximity to the myocardial region;
wherein the hydrogel agent comprises alginate polymers and peptides covalently bonded to the alginate polymers.

129-130. (canceled)
